(19) United States
(12) Patent Application Publication
(10) Pub. No.: US 2026/0157981 A1
(43) Pub. Date: Jun. 11, 2026

(54) Compositions Comprising Fingolimod or a Salt Thereof

(71) Applicant: Azurity Pharmaceuticals Ireland Limited, Dublin 4 (IE)

(72) Inventors: Sushant Omprakash Dube, Mumbai (IN); Pramod Ramdas Parshuramkar, Pune (IN); Mansing Bapusaheb Patil, Sangli (IN); Pankaj Kisan Chatki, Hyderabad (IN); Satheesh Balasubramanian, Hyderabad (IN); Sumitra Ashokkumar Pillai, Hyderabad (IN)

(73) Assignee: Azurity Pharmaceuticals Ireland Limited, Dublin 4 (IE)

(21) Appl. No.: 19/409,902

(22) Filed: Dec. 5, 2025

(30) Foreign Application Priority Data
Dec. 7, 2024 (IN) 202411096780
Jun. 24, 2025 (IN) 202511060413
Aug. 1, 2025 (IN) 202511073529

(51) Int. Cl. A61K 31/137; A61K 9/08; A61K 47/02; A61K 47/10; A61K 47/12; A61K 47/14; A61K 47/18; A61K 47/32; A61K 47/36; A61K 47/38

(52) U.S. Cl. CPC A61K 31/137; A61K 9/08; A61K 47/02; A61K 47/10; A61K 47/12; A61K 47/14; A61K 47/18; A61K 47/183; A61K 47/32; A61K 47/36; A61K 47/38

(57) ABSTRACT

The present disclosure relates to novel stable liquid compositions comprising fingolimod, or a pharmaceutically acceptable salt thereof. The disclosure also relates to kits for preparing liquid compositions comprising fingolimod, or a pharmaceutically acceptable salt thereof, as well as methods of treating a subject with multiple sclerosis (MS) using the compositions.

### CROSS REFERENCE TO RELATED APPLICATIONS

This application claims the benefit of priority of Indian Provisional Application No. 202411096780, filed Dec. 7, 2024, Indian Provisional Application No. 202511060413, filed Jun. 24, 2025, and Indian Provisional Application No. 202511073529, filed Aug. 1, 2025, each of which is incorporated by reference herein in its entirety for any purpose.

### FIELD

The present disclosure relates to novel stable liquid compositions comprising fingolimod, or a pharmaceutically acceptable salt thereof. The disclosure also relates to kits for preparing liquid compositions comprising fingolimod, or a pharmaceutically acceptable salt thereof, as well as methods of treating a subject with multiple sclerosis (MS) using the compositions.

### BACKGROUND

Multiple sclerosis (MS) is a chronic disorder of the central nervous system that disrupts the normal functioning of the brain, optic nerves, and spinal cord through inflammation and tissue loss. The location of the inflammation varies from patient to patient, and from episode to episode, resulting in a variety of clinical symptoms.

Commercially available MS drugs are often delivered by frequent injections, either intravenously or intra-muscularly, varying from once-per-day to once-per-week depending on the drug. Gilenya® is an oral medication that was developed and approved for the treatment of MS. The active ingredient in Gilenya® is fingolimod hydrochloride (structure below).

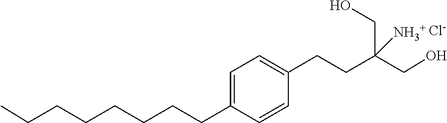 [SMILES: CCCCCCCCc1ccc(CCC([NH3+])(CO)CO)cc1 ; [Cl-]]

Fingolimod hydrochloride (i.e., 2-amino-2-[2-(4-octylphenyl)ethyl]propane-1,3-diol hydrochloride; FTY720) is a sphingosine 1-phosphate (S1P) receptor modulator and is believed to reduce the number of lymphocytes circulating in the blood stream by reversibly trapping a proportion of them in the lymph nodes. Consequently, the number of activated lymphocytes reaching the brain decreases, resulting in reduced inflammatory destruction caused by MS (see, e.g., “FTY720 therapy exerts differential effects on T cell subsets in multiple sclerosis” Mehling, M., et al., Neurology, 2008 Oct. 14, 71(16):1261-7; and “Oral fingolimod (FTY720) for relapsing multiple sclerosis” Kappos, L., et al., FTY720 D2201 Study Group. N Engl J. Med., 2006 Sep. 14, 355(11):1124-40).

Gilenya®, which is sold as capsules containing fingolimod hydrochloride, became the first oral drug product approved to treat relapsing forms of MS, including clinically isolated syndrome, relapsing-remitting disease, and active secondary progressive disease, in patients 10 years of age and older.

Unfortunately, it is estimated that 50% of the population have problems swallowing tablets or capsules (see, e.g., “Drug-delivery products and the Zydis fast-dissolving dosage form,” Seager, Journal of Pharmacol. and Pharm., 1998, 50(4):375-382). For example, capsule dosage forms become sticky when wetted by saliva, and if the patient experiences difficulty in swallowing the capsule on the first attempt, then the capsule must often be discarded. These issues may lead to poor patient compliance or even noncompliance.

Further, there are approximately 5,000 pediatric patients with MS. It is especially hard to medicate children who often have difficulty swallowing or are unwilling to swallow tablets or capsules. Again, this may lead to poor patient compliance or even noncompliance.

In addition, dysphagia, or difficulty swallowing based on a decline in muscle strength, is commonly experienced by MS patients and is reported in about 90% of the MS patient population with about 50% of the MS patient population suffering from moderate to severe dysphagia. Among the clinical forms of MS, the progressive forms (primary progressive and secondary progressive) were more frequently associated with severe dysphagia, while the relapsing-remitting form presented more often mild and moderate dysphagia.

Dysphagia can reduce the quality of life and increase the risk of dehydration, which in turn leads to increased mortality in the last stages of the disease. Dysphagia is associated with a 13% increase in mortality and causes 60,000 deaths per year in the U.S. due to complications, especially via aspiration pneumonia.

An oral liquid medication would be extremely beneficial for patients who have trouble swallowing pills or prefer a liquid formulation (i.e., a liquid composition). Fingolimod exhibits significant stability challenges in liquid or aqueous media, primarily due to its susceptibility to photolytic, thermal, basic and oxidative degradation. For example, even Novartis, the manufacturer of Gilenya®, has noted that aqueous liquid compositions of S1P receptor modulators are unstable, with crystalline deposits of the drug developing either shortly after preparation or upon storage. See US 2008/0194526 at para. [0109]. To date, despite efforts by Novartis and others, there is still no FDA-approved liquid composition of fingolimod.

In addition to there being a need for an oral liquid composition of fingolimod, there is a need for a ready to use dosage form. Ready to use dosage forms are preferred when compared to a dosage form that a pharmacist or patient must prepare, for example by mixing different components together to create the final dosage form, because a ready to use dosage form eliminates the potential for contamination during preparation as well as the potential for dose fluctuation if the product is not prepared properly. Accordingly, there is a need for a ready to use oral dosage form.

The present disclosure resolves the above concerns by providing liquid pharmaceutical compositions of fingolimod for oral administration which are safe, therapeutically effective, ready to administer, palatable, and stable for extended periods of time, under varying storage conditions.

### SUMMARY

In an aspect, the present disclosure relates to oral liquid pharmaceutical compositions comprising: (a) fingolimod or a pharmaceutically acceptable salt thereof at a concentration of about 0.01 mg/mL to about 10 mg/mL; (b) a pharmaceutically acceptable vehicle; and (c) optionally, at least one pharmaceutically acceptable excipient.

In another aspect, the present disclosure relates to oral liquid pharmaceutical compositions comprising fingolimod or a pharmaceutically acceptable salt thereof. In another aspect, the present disclosure relates to oral liquid pharmaceutical compositions comprising fingolimod hydrochloride.

In an aspect, the present disclosure relates to oral liquid pharmaceutical compositions comprising: (a) fingolimod or a pharmaceutically acceptable salt thereof at a concentration of about 0.05 mg/mL to about 10 mg/mL; (b) a pharmaceutically acceptable vehicle; and (c) optionally, at least one pharmaceutically acceptable excipient.

In another aspect, the present disclosure relates to oral liquid pharmaceutical compositions comprising: (a) fingolimod or a pharmaceutically acceptable salt thereof at a concentration of about 0.05 mg/mL to about 10 mg/mL; (b) a pharmaceutically acceptable vehicle; and (c) optionally, at least one pharmaceutically acceptable excipient selected from a group consisting of preservatives, sweetening agents, pH adjusting agents such as an alkalizer, buffering agents, crystal growth inhibitors (i.e., binders), complexing agents, thickening agents (i.e., a viscosity agent), anti-oxidants, flavoring agents, stabilizers, solubilizers, coloring agents, flavoring agents, and mixtures thereof.

In an aspect, the present disclosure relates to oral liquid pharmaceutical compositions comprising: (a) fingolimod or a pharmaceutically acceptable salt thereof at a concentration of about 0.05 mg/mL to about 10 mg/mL; (b) a pharmaceutically acceptable vehicle; (c) at least one preservative; (d) optionally at least sweetening agent; (e) at least one pH adjusting agent; (f) at least one buffering agent; (g) at least one crystal growth inhibitor and optionally, at least one pharmaceutically acceptable excipient.

In an aspect, the present disclosure relates to oral liquid pharmaceutical compositions comprising fingolimod or a pharmaceutically acceptable salts thereof, wherein the concentration of fingolimod in the composition is about 0.1 mg/mL.

In another aspect, the present disclosure relates to oral liquid compositions comprising fingolimod or a pharmaceutically acceptable salt thereof and pharmaceutically acceptable vehicle; wherein the pharmaceutically acceptable vehicle is selected from a group consisting of water, purified water, isopropyl alcohol, methanol, acetone, ethanol, 1-propanol, and butanediol or combinations thereof.

In another aspect, the present disclosure relates to oral liquid pharmaceutical compositions comprising fingolimod or a pharmaceutically acceptable salt thereof and at least one pharmaceutically acceptable solvent, wherein said solvent is not propylene glycol.

In an aspect, the present disclosure relates to oral liquid pharmaceutical compositions comprising fingolimod or a salt thereof, at least one preservative and at least one pH adjuster.

In another aspect, the present disclosure relates to oral liquid pharmaceutical compositions comprising fingolimod or a salt thereof and at least one pH adjusting agent selected from HCl and NaOH or combination thereof.

In an aspect, the present disclosure relates to oral liquid pharmaceutical compositions comprising: (a) fingolimod or a pharmaceutically acceptable salt thereof at a concentration of about 0.1 mg/mL or more; (b) a pharmaceutically acceptable vehicle; (c) at least one pharmaceutically acceptable excipient, wherein the pH of pharmaceutical composition ranges from about 3 to about 6.

In another aspect, the present disclosure relates to oral liquid pharmaceutical compositions as described above, wherein the pH of pharmaceutical composition ranges from about 4 to about 6.

In another aspect, the present disclosure relates to oral liquid pharmaceutical compositions comprising fingolimod or a pharmaceutically acceptable salt thereof, wherein the pH of said composition is about 4.5.

In an aspect, the present disclosure relates to oral liquid pharmaceutical compositions comprising fingolimod or a pharmaceutically acceptable salt thereof, wherein the composition is buffer-free.

In an aspect, the present disclosure relates to oral liquid pharmaceutical compositions comprising fingolimod or a pharmaceutically acceptable salt thereof, wherein the composition is substantially free of buffer.

In an aspect, the present disclosure relates to oral liquid pharmaceutical compositions comprising fingolimod or a pharmaceutically acceptable salt thereof, wherein the composition is free of citric acid buffer.

In an aspect, the present disclosure relates to oral liquid pharmaceutical compositions comprising fingolimod or a pharmaceutically acceptable salt thereof, wherein the composition is substantially free of citric acid buffer.

In an aspect, the present disclosure relates to oral liquid pharmaceutical compositions comprising fingolimod or a pharmaceutically acceptable salt thereof, wherein the composition is free of phosphate buffer.

In an aspect, the present disclosure relates to oral liquid pharmaceutical compositions comprising fingolimod or a pharmaceutically acceptable salt thereof, wherein the composition is substantially free of phosphate buffer.

In an aspect, the present disclosure relates to oral liquid pharmaceutical compositions comprising fingolimod or a pharmaceutically acceptable salt thereof, wherein the composition is free of acetate buffer.

In an aspect, the present disclosure relates to oral liquid pharmaceutical compositions comprising fingolimod or a pharmaceutically acceptable salt thereof, wherein the composition is substantially free of acetate buffer.

In an aspect, the present disclosure relates to oral liquid pharmaceutical compositions comprising fingolimod or a pharmaceutically acceptable salt thereof, wherein the composition is substantially free of a complexing agent.

In an aspect, the present disclosure relates to oral liquid pharmaceutical compositions comprising fingolimod or a pharmaceutically acceptable salt thereof, wherein the composition is free of a complexing agent.

In an aspect, the present disclosure relates to oral liquid pharmaceutical compositions comprising fingolimod or a pharmaceutically acceptable salt thereof, wherein the composition is substantially free of cyclodextrin or derivatives thereof.

In an aspect, the present disclosure relates to oral liquid pharmaceutical compositions comprising fingolimod or a pharmaceutically acceptable salt thereof, wherein the composition is free of cyclodextrin or derivatives thereof.

In an aspect, the present disclosure relates to oral liquid pharmaceutical compositions comprising fingolimod or a pharmaceutically acceptable salt thereof in the form of an aqueous or non-aqueous solution.

In an aspect, the present disclosure relates to oral liquid pharmaceutical compositions comprising: (a) fingolimod or a pharmaceutically acceptable salt thereof at a concentration of about 0.1 mg/mL or more; (b) a pharmaceutically acceptable vehicle; and (c) optionally, at least one pharmaceutically acceptable excipient, wherein the pharmaceutical composition is stable for at least 12 months at about 25° C. and about 60% Relative Humidity (RH).

In an aspect, the present disclosure relates to oral liquid pharmaceutical compositions comprising: (a) fingolimod or a pharmaceutically acceptable salt thereof at a concentration of about 0.1 mg/mL or more; (b) a pharmaceutically acceptable vehicle; (c) a pH adjusting agent; (d) a preservative; and (e) optionally, a sweetening agents and (f) optionally, at least one other pharmaceutically acceptable excipient, wherein the pharmaceutical composition is stable for at least 12 months at about 25° C. and about 60% RH.

In an aspect, the present disclosure relates to oral liquid pharmaceutical compositions comprising: (a) fingolimod or a pharmaceutically acceptable salt thereof at a concentration of about 0.1 mg/mL or more; (b) a pharmaceutically acceptable vehicle; and (c) optionally, at least one pharmaceutically acceptable excipient, wherein the pharmaceutical composition is stable for at least 6 months at about 25° C. and about 40% RH.

In an aspect, the present disclosure relates to oral liquid pharmaceutical compositions comprising: (a) fingolimod or a pharmaceutically acceptable salt thereof at a concentration of about 0.1 mg/mL or more; (b) a pharmaceutically acceptable vehicle; and (c) optionally, at least one pharmaceutically acceptable excipient, wherein the pharmaceutical composition is stable for at least 6 months at about 40° C. and about 75% RH.

In an aspect, the present disclosure relates to oral liquid pharmaceutical compositions comprising: (a) fingolimod or a pharmaceutically acceptable salt thereof at a concentration of about 0.1 mg/mL or more; (b) a pharmaceutically acceptable vehicle; (c) a pH adjusting agent; (d) a preservative; and (e) optionally, a sweetening agents and (f) optionally, at least one other pharmaceutically acceptable excipient, wherein the pharmaceutical composition is stable for at least 6 months at about 40° C. and about 75% RH.

In an aspect, the present disclosure relates to oral liquid pharmaceutical compositions comprising fingolimod or a pharmaceutically acceptable salt thereof, wherein the composition is stable at room temperature for at least 3 months, at least 6 months, or at least 12 months.

In an aspect, the liquid composition is free of precipitates. In another aspect, the liquid composition is free of visible particulates. In yet another aspect, the liquid composition is free of any crystalline deposits. In an aspect, the liquid composition is substantially free of precipitates. In another aspect, the liquid composition is substantially free of visible particulates. In yet another aspect, the liquid composition is substantially free of any crystalline deposits.

In an aspect, the present disclosure relates to oral liquid pharmaceutical compositions comprising fingolimod or a pharmaceutically acceptable salt thereof, wherein the composition is a stable aqueous solution.

In an aspect, the present disclosure relates to oral liquid pharmaceutical compositions comprising fingolimod or a pharmaceutically acceptable salt thereof, wherein the composition is stable after at least one freeze-thaw cycle.

In another aspect, the present disclosure relates to oral liquid pharmaceutical compositions comprising fingolimod or a pharmaceutically acceptable salt thereof, wherein the composition is photostable.

In another aspect, the present disclosure relates to oral liquid pharmaceutical compositions comprising fingolimod or a pharmaceutically acceptable salt thereof, wherein the composition is a photostable aqueous solution.

In an aspect, the present disclosure relates to oral liquid pharmaceutical compositions comprising fingolimod or a pharmaceutically acceptable salt thereof, wherein the composition is ready to use.

In an aspect, the present disclosure relates to stable ready-to-administer (RTA) and safe-to-administer (STA) oral liquid pharmaceutical compositions comprising fingolimod or a pharmaceutically acceptable salt thereof suitable for oral administration.

In an aspect, the present disclosure relates to a kit comprising:
(i) a first container comprising an oral liquid pharmaceutical composition as described herein; and
(ii) a second container comprising a pharmaceutically acceptable vehicle.

In another aspect, the present disclosure relates to a kit comprising:
(i) a first container comprising fingolimod or a pharmaceutically acceptable salt thereof; and
(ii) a second container comprising a pharmaceutically acceptable vehicle.

In an aspect, the present disclosure relates to a method of treating a subject with multiple sclerosis (MS) comprising administering to the subject in need thereof an oral liquid pharmaceutical composition as described herein.

In another aspect, the present disclosure relates to a method of treating a subject with multiple sclerosis (MS), the method comprising combining the contents of the first container and the contents of the second container in a kit as described herein and administering the resultant combination to the subject.

In an aspect, the present disclosure relates to a method of preparing an oral liquid pharmaceutical composition as described herein.

Additional objects and advantages will be set forth, in part, in the description that follows, and, in part, will be understood from the description or learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

### DETAILED DESCRIPTION

### Definitions

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art. Standard techniques may be used for preparation and analysis of solid forms. As appropriate, procedures involving the use of commercially available kits and reagents are generally carried out in accordance with manufacturer-defined protocols and conditions unless otherwise noted.

While the following text may reference or exemplify specific embodiments, it is not intended to limit the scope of the disclosure.

The articles “a,” “an,” and “the” as used herein refers to “one or more” or “at least one,” unless otherwise indicated. That is, reference to any element or component of an embodiment by the indefinite article “a,” “an,” or “the” does not exclude the possibility that more than one element or component is present.

The term “about” as used herein indicates and encompasses an indicated value and a range above and below that value. In certain embodiments, the term “about” indicates the designated value±20%, ±10%, ±5%, or ±1%. In certain embodiments, where applicable, the term “about” indicates the designated value(s)±one standard deviation of that value(s). When the term “about” precedes a list of numerical values or ranges, the terms modify all of the values or ranges provided in the list. In some embodiments, the term “about” may include numerical values that are rounded to the nearest significant figure.

The term “comprising”, as used herein, also specifically includes embodiments “consisting of” and “consisting essentially of” the recited elements, unless specifically indicated otherwise.

The term “concentration” when used herein in reference to fingolimod or a pharmaceutically acceptable salt thereof should be understood to be referring to the concentration of free base, rather than the salt. For example, a formulation may be prepared using 0.112 mg of fingolimod hydrochloride, but the final liquid composition would be said to have a concentration of 0.1 mg. In some instances, the present disclosure refers to “concentration” while in other instances the disclosure refers to “an amount of fingolimod or salt thereof that is equivalent to” an amount of fingolimod. These should be understood to mean the same thing—i.e., the amount of fingolimod free base in the final liquid composition.

The term “active ingredient” or “active pharmaceutical ingredient” or “API” as used herein refers to a compound (e.g., fingolimod) that can be used for treating a disorder or condition in a subject (e.g., a patient) or for preventing one or more symptoms of such disorder or condition in the subject.

The terms “effective amount” or “therapeutically effective amount,” as used herein, generally refers to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, in some embodiments, an “effective amount” for therapeutic uses is the amount of a compound required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. In some embodiments, an “effective amount” of a compound disclosed herein is an amount effective to achieve a desired pharmacologic effect or therapeutic improvement without undue adverse side effects. It is understood that “an effect amount” or “a therapeutically effective amount” can vary from subject to subject, due to variation in metabolism of the active compound, age, weight, general condition of the subject, the condition being treated, the severity of the condition being treated, and the judgment of the prescribing physician. By way of example only, therapeutically effective amounts may be determined by routine experimentation, including but not limited to a dose escalation clinical trial.

The term “bioequivalence” or “bioequivalent” as used herein refers to two compositions, dosage forms, or products of an active ingredient having biological equivalence. It is generally considered bioequivalent if the 90% Confidence Interval (“CI”) of the relative mean Cmax, AUC(0-t) and AUC(0-∞) of the test composition to the reference composition is within 80.0% to 125.0% in the fasting or fed state of a tested subject.

The term “fingolimod” as used herein refers to the free base form of the molecule and includes polymorphs, stereoisomers, and mixtures thereof. The terms “pharmaceutically acceptable salt thereof,” “salt thereof,” and the like, when used in reference to fingolimod, refers to any pharmaceutically acceptable salt. For example, in some embodiments, the salt may be a salt with an inorganic acid, such as hydrochloride, hydrobromide, and sulfate. In some embodiments, the salt may be a salt with an organic acid, such as acetate, fumarate, maleate, benzoate, citrate, malate, methanesulfonate, and benzenesulfonate salts. In some embodiments, the salt may be a salt with a metal, such as sodium, potassium, calcium, and aluminum.

The terms “composition,” “pharmaceutical composition,” and “formulation” are used interchangeably herein, and refer to a combination of two or more ingredients. For example, a composition may comprise an active ingredient (e.g., fingolimod) and a pH adjusting agent (e.g., hydrogen chloride). In some embodiments, the composition is in the form of a liquid.

The term “excipient” as used herein refers to any inactive substance in a composition (e.g., swelling agent, controlling-release agent, osmotic agent). An excipient may provide, without limitation, bulk, consistency, stability, binding ability, lubrication, disintegrating ability, etc., to the composition comprising an active ingredient. An excipient may function for multiple purposes.

The terms “include,” “such as,” and the like, as used herein, are intended to convey inclusion without limitation, unless otherwise specifically indicated.

The term “powder” as used herein refers to any composition or formulation which is dry and flowable. Non-limiting examples include granules, flakes, and spheroids. In some embodiments, the powder can be prepared and mixed with a liquid to provide a solution.

The term “liquid” as used herein, when used in the context of a composition, refers to a composition that is flowable but not dry. For example, a liquid composition may be a solution, a suspension, or an emulsion.

The term “solution” as used herein refers to a homogenous mixture of two or more substances. In some embodiments, the solution is clear. In some embodiments, the solution is clear and colorless. In some embodiments, the liquid composition is a clear solution under acidic conditions. In some embodiments, the solution contains no precipitate. In some embodiments, the solution contains no visible particles. In some embodiments, the solution contains no crystalline deposits.

The term “area under curve (AUC)” or “total plasma exposure” as used herein, expressed in μg·hr/mL, refers to the total integrated area under plasma level time profile and expresses the total amount of the active ingredient that comes into systemic circulation after administration.

The term “alkalizer” as used herein refers to a substance that neutralizes or reduces acidity. In some embodiments, the alkalizer is meglumine or L-Arginine.

The term “anti-caking agent” as used herein refers to an excipient used to prevent aggregation of particles. Examples of anti-caking agents include silicon dioxide, magnesium silicate, and ammonium chloride.

The term “antifoaming agent” as used herein refers to an excipient used to prevent or break up foam in pharmaceutical processes and products. Examples of antifoaming agents include insoluble oils (such as castor oil), polydimethylsiloxanes (such as simethicone) and other silicones, certain alcohols (such as cetostearyl alcohol), stearates, and glycols.

The term “binder” as used herein refers to an excipient used to improve the cohesion of APIs and excipients in a dosage form. The term “crystal growth inhibitor” as used herein refers to an agent or additive that is capable of reducing or eliminating the increase in crystal size or particle size of an active ingredient within a composition during storage. The terms “binder” and “crystal growth inhibitor” may be used interchangeably herein and would be understood by one skilled in the art to encompass the same excipients and functions. In some embodiments, the binder (or crystalline growth inhibitor) is hypromellose or povidone. In some embodiments, the binder (or crystalline growth inhibitor) includes hypromellose or povidone or combination thereof. In some embodiments, the binder/crystal growth inhibitor is hypromellose. In some embodiments, the binder/crystal growth inhibitor is povidone.

The term “buffering agent” as used herein refers to an excipient used to maintain the pH of a composition. Examples of buffering agents include salts of weak acids and bases as well as weak acids, such as citric acid. In some embodiments, the buffering agent may be a citrate buffer, acetate buffer, phosphate buffer, bicarbonate buffer and ammonium buffer.

The phrases “buffer-free” and “free of buffer” refer to compositions that comprise no buffers. The pharmaceutical compositions of the present invention are optionally buffer-free compositions.

The phrase “substantially buffer-free” refers to compositions that contain an insignificant or negligible quantity of a buffer(s).

The terms “carrier,” “vehicle,” “pharmaceutically acceptable vehicle,” “solvent” and the like are used interchangeably herein and refer to an excipient that delivers an active ingredient or composition or dissolves another substance to form a homogeneous solution. Examples of vehicle include water, such as purified water, alcohols, glycols, dimethylacetamide N-methylpyrollidone, dimethyl sulfoxide, ringer's solution, isotonic sodium chloride solution, glycerine, alcohols, propylene glycol, ethanol, polyethylene glycol, or their mixtures thereof. Carriers can help formulate an active ingredient or composition into a dosage form such as tablets, pills, dragees, capsules, liquids, gels, syrups, slurries, suspensions, and the like, for administration to a subject in need thereof. In some embodiments, the carrier can be water or an aqueous solution containing other excipients.

The term “complexing agent” as used herein refers to an excipient that is used as an additive to form a stable association (a coordination complex or an inclusion complex) with a therapeutic agent (the drug) or with metal ions present in a formulation. Examples of complexing agents include Ethylenediaminetetraacetic acid (EDTA), citric acid, tartaric acid, beta-cyclodextrin, alpha-cyclodextrin, sulfobutyl ether beta-cyclodextrin (SBEBCD), carboxymethyl-gamma-cyclodextrin or combination thereof. The phrase “free of a complexing agent” refers to formulations that do not contain a complexing agent. The phrase “substantially free of a complexing agent” refers to formulations that contain an insignificant or negligible quantity of the complexing agent.

The term “flavoring agent” as used herein refers to an excipient that gives a composition or dosage form an additional taste or flavor. For example, flavoring agents may help in masking unpleasant tastes of APIs or excipients. In some embodiments, the flavoring agent is grape flavoring. In some embodiments, a flavoring agent may be added by a pharmacist or a patient.

The terms “pH adjuster” and “pH adjusting agent” are used interchangeably herein and refer to an excipient used to regulate the pH of a composition. Examples of pH adjusters include sodium hydroxide, hydrochloric acid, phosphoric acid, and disodium fumarate. In some embodiments, the pH adjuster is at a concentration of about 0.1 N. In some embodiments, the pH adjuster is an alkalizer.

The term “preservative” as used herein refers to an excipient used to preserve the integrity of the composition. For example, in some embodiments, a preservative is used to slow down or stop degradation of the active ingredient or the other excipients in a composition and/or to prevent bacterial growth. Examples of preservatives include, but are not limited to, alcohols (e.g., benzyl alcohol), parabens (e.g., methyl paraben, propyl paraben) and salts thereof, sodium propionate (e.g., sodium propionate anhydrous), benzalkonium chloride, benzyl benzoate, chlorobutanol, sodium benzoate, boric acid, benzoic acid, formaldehyde, and phenols.

The term “stabilizer” as used herein refers to an excipient that inhibits, prevents, slows down, or reduces the degradation of fingolimod or a composition thereof. Examples of stabilizers include, but are not limited to, amino acids such as glycine, alanine, glutamate, sodium glutamate, L-arginine, lysine, L-cysteine and methionine; sodium chloride or sodium sulfate salts; ethylenediaminetetraacetic acid (EDTA), metal ions such as zinc, magnesium and calcium or mixtures thereof; natural or synthetic gums; cellulosic derivatives such as carboxy methyl cellulose, ethyl cellulose, hydroxyethyl cellulose, hydroxyl propyl cellulose, hydroxyl propyl methylcellulose, methyl cellulose, and polyanionic cellulose; sugars; sugar alcohols; monosaccharides, disaccharides or polysaccharides; or combinations thereof. In some embodiments, the concentration of stabilizer in the liquid compositions described herein ranges from about 0.001% to about 20% w/w, based on total weight of the composition.

The term “packaged product” refers to a product (such as a pharmaceutical composition) that is in a container. For example, in some embodiments, the packaged product comprises a liquid composition in a container. In some embodiments, the container is chosen from a bottle, a vial, a pouch, a bag, and a box. In some embodiments, the container reduces exposure of the contents (e.g., the liquid composition) to light. In some embodiments, a packaged product may be further packaged. For example, in some embodiments, the packaged product comprises a liquid composition in a container, which is then placed into another container such as a box (such as a monocarton).

The term “fill volume” refers to the volume of space a product takes up within a container, rather than the total capacity (which includes the space between the contents of a container and the closure) of the container.

The terms “release,” “released”, “releasing”, and the like, when used in connection with a pharmaceutical composition or dosage form, refer to the process or the portion of the active ingredient that leaves the composition or dosage form. The results of the dissolution testing may be reported as % (e.g., % w/w) released as a function of time or as the release time. In some embodiments, “complete release” of an active ingredient occurs when at least 90% of the active ingredient has been released from the dosage form. Dissolution testing may be performed in accordance with the United States Pharmacopoeia (USP) dissolution procedure. Dissolution testing may also generate drug release profiles.

The term “stable” as used herein refers to the ability of a composition or substance to resist undergoing chemical reactions or decomposing under specified conditions. In general, a stable composition or compound is one that maintains its structure and properties without readily reacting with other substances in its environment. For example, in some embodiments, a composition is stable if no precipitation occurs when the composition is subjected to certain specified conditions. In some embodiments, a composition is stable if no visible particulates form when the composition is subjected to certain specified conditions. In some embodiments, a composition is stable if it is free of crystalline deposits when the composition is subjected to certain specified conditions. In some embodiments, a composition is stable if there is no discoloration observed when the composition is subjected to certain specified conditions. In some embodiments, a composition is stable if the active pharmaceutical ingredient in the composition does not degrade, or degrades only slightly, when the composition is subjected to certain specified conditions. In some embodiments, a composition is stable if at least 80% of the active pharmaceutical ingredient in the composition remains when the composition is subjected to certain specified conditions. In some embodiments, a composition is stable if a minimal amount of impurities form when the composition is subjected to certain specified conditions. In some embodiments, the composition is shelf stable. The term “stable” indicates chemical and/or physical stability.

In some embodiments, a composition is stable if it is photostable. As used herein, “photostable” or “photostability” refers to the ability of a chemical, material, or product to withstand exposure to light. In some embodiments, a photostable composition can withstand exposure to an integrated near ultraviolet energy and/or illumination. In some embodiments, a photostable composition is resistant to photochemical reactions. In some embodiments, a photostable composition is not photolabile.

In some embodiments, a stable composition is one that has undergone a freeze-thaw cycle without adverse effects. As used herein, the term “freeze-thaw cycle” refers to a process of subjecting a sample to alternating freezing and thawing conditions.

The term “precipitate” refers to a solid formed by a change in a liquid, often due to a chemical reaction or change in temperature that decreases solubility of the solid. The term “precipitate” can also refer to the process of sedimentation of a solid material from a liquid composition. In some embodiments, the precipitate is a salt of the API. In some embodiments, the precipitate is a crystalline deposit of the API. In some embodiments, the precipitate is a crystalline deposit of the API and a salt of the API.

The terms “ready-to-use,” “ready to use,” “ready to administer” or “RTU” as used herein are used interchangeably and refer to a composition that is available to administer without any further dilution and/or manipulation. In some embodiments, the liquid composition does not require dilution prior to administration.

The term “beyond use date” or “BUD” as used herein refers to the date or period of time after which a medication should no longer be used. The date or timeframe is typically determined by the date on which the medication was compounded. In some embodiments, the recommended beyond use date (BUD) of the compositions disclosed herein is at least about 30 days. In some embodiments, the recommended BUD of the compositions disclosed herein is at least about 60 days. In some embodiments, the recommended BUD of the compositions-disclosed herein is at least about 90 days.

The term “after storage” as used herein, in the context of analyzing a composition, refers to analyzing the composition after any period of time after the initial preparation of the composition. For example, “after storage” as used herein, without limitation, can refer to analyzing a composition after keeping the composition at about 40° C. and about 75% relative humidity for a period of time. In some embodiments, the composition is kept at about 25° C. and about 60% relative humidity for up to about 24 months. In some embodiments, the composition is kept at about 40° C. and about 75% relative humidity for at least about 1 month. In some embodiments, the composition is kept at about 40° C. and about 75% relative humidity for at least about 3 months. In some embodiments, the composition is kept at about 40° C. and about 75% relative humidity for up to about 24 months. In some embodiments, the composition is kept at about 60° C. for at least about one month. In some embodiments, the composition is kept at about 30° C. and about 65% relative humidity for a period of time. In some embodiments, the composition is kept at about 30° C. and about 65% relative humidity for at least 12 months.

The term “sweetener” or “sweetening agent” as used herein refers to an excipient used to improve the taste of pharmaceutical compositions. Examples of sweeteners include sucralose, glucose, sorbitol, aspartame, and saccharin sodium. In some embodiments, the sweetener is sucralose or xylitol. In some embodiments, the sweetener is sucralose. In some embodiments, the sweetener is xylitol.

The terms “treat,” “treating,” “treatment,” and the like, as used herein, refer to attaining a beneficial or desired result, such as a clinical result. In some embodiments, the beneficial or desired result is any one or more of the following: inhibiting or suppressing the onset or development of a condition, reducing the severity of the condition, reducing the number or severity of symptoms associated with the condition, increasing the quality of life of a patient suffering from the condition, decreasing the dose of another medication required to treat the condition, enhancing the effect of another medication a patient is taking for the condition, and prolonging the survival of a patient having the condition. In some embodiments, treatment refers to management of the disease and/or its symptoms. In some embodiments, treatment refers to prophylaxis treatment.

The term “subject” as used herein refers to a mammal, such as an animal or a human. Hence, the methods disclosed herein can be useful in human therapy and veterinary applications. In one embodiment, the subject is an animal. In another embodiment, the subject is a human.

The term “patient compliance” refers to the degree to which a patient correctly follows medical advice.

The term “pediatric” as used herein refers to the branch of medicine dealing with children and their diseases. In some embodiments, a pediatric patient is about 10 years old to about 18 years old.

The term “geriatric” as used herein refers to the branch of medicine that deals with the problems and diseases of old age, and the medical care and treatment of aging people. In some embodiments, a geriatric patient is at least 65 years old.

At various places in the present disclosure, variables or parameters are disclosed in groups or ranges. It is specifically intended that the description include each and every individual sub combination of the members of such groups and ranges. For example, an integer in the range of 0 to 10 is specifically intended to individually disclose 0, 1, 2, 3, 4, 5, 6, 7, 8, 9, and 10.

### Compositions, Kits, Methods and Uses Thereof

In some embodiments, the present disclosure relates to fingolimod, salts, compositions, kits, and methods and uses thereof.

### Oral Liquid Pharmaceutical Compositions

In some embodiments, the present disclosure relates to a liquid composition comprising fingolimod or a salt thereof.

In one embodiment, the present disclosure relates to an oral pharmaceutical composition comprising: (a) fingolimod or a pharmaceutically acceptable salt thereof at a concentration of about 0.05 mg/mL to about 10 mg/mL; (b) a pharmaceutically acceptable vehicle; and (c) optionally, at least one pharmaceutically acceptable excipient.

In one embodiment, the present disclosure relates to an oral pharmaceutical composition comprising: (a) fingolimod or a pharmaceutically acceptable salt thereof at a concentration of about 0.05 mg/mL to about 10 mg/mL; (b) a pharmaceutically acceptable vehicle; (c) at least one preservative; (d) optionally, at least one sweetening agent; (e) at least one pH adjusting agent; (f) at least one buffering agent; and (g) optionally at least one pharmaceutically acceptable excipient selected from a group consisting of complexing agents, thickening agents, anti-oxidants, flavoring agents, solubilizers, coloring agents and mixtures thereof.

In one embodiment, the present disclosure relates to an oral pharmaceutical composition comprising: (a) fingolimod or a pharmaceutically acceptable salt thereof at a concentration of about 0.05 mg/mL to about 10 mg/mL; (b) a pharmaceutically acceptable vehicle; and (c) at least one preservative; (d) optionally, at least one sweetening agent; (e) at least one pH adjusting agents; (f) at least one buffering agent and (g) optionally at least one pharmaceutically acceptable excipient.

In an embodiment, the present disclosure relates to an oral pharmaceutical solution comprising (a) fingolimod or a pharmaceutically acceptable salt thereof at a concentration of about 0.1 mg/mL to about 10 mg/mL; (b) pharmaceutically acceptable vehicle; and (c) at least one other pharmaceutically acceptable excipient selected from a group consisting of solubilizers, pH adjusting agents, thickening agents, anti-oxidants, preservatives, flavoring agents, sweetening agents, and mixtures thereof, wherein said solution is substantially buffer-free and has pH of about 3 to about 6.

In an embodiment, the present disclosure relates to an oral liquid pharmaceutical composition comprising:
(a) about 0.001% to about 0.1% w/w of fingolimod or a pharmaceutically acceptable salt thereof;
(b) about 0.01% to about 3% w/w of at least one preservative;
(c) optionally, about 0.01% to about 3% w/w of at least one sweetening agent;
(d) about 0.1% to about 5% w/w of at least one pH adjusting agent;
(e) about 10% to about 99.9% w/w of a pharmaceutically acceptable vehicle; and
(f) optionally at least one pharmaceutically acceptable excipient selected from buffering agents, complexing agents, binders (i.e., crystal growth inhibitors), and antioxidants;
   wherein the pH of the composition ranges from about 3 to about 6.

In another embodiment, the present disclosure relates to an oral liquid solution comprising:
(a) about 0.001% to about 0.1% w/w of fingolimod or a pharmaceutically acceptable salt thereof;
(b) about 0.01% to about 3% w/w of at least one preservative;
(c) optionally, about 0.01% to about 3% w/w of at least one sweetening agent;
(d) about 0.1% to about 5% w/w of at least one pH adjusting agent;
(e) about 10% to about 99.9% w/w of a pharmaceutically acceptable vehicle; and
(f) optionally at least one pharmaceutically acceptable excipient selected from buffering agents, complexing agents, binders (i.e., crystal growth inhibitors), and antioxidants;
wherein the pH of the composition ranges from about 3 to about 6.

In an embodiment, the present disclosure relates to an oral liquid composition comprising:
(a) about 0.001% to about 0.1% w/w of fingolimod or a pharmaceutically acceptable salt thereof;
(b) about 0.01% to about 3% w/w of at least one preservative;
(c) optionally, about 0.01% to about 3% w/w of at least one sweetening agent;
(d) about 0.1% to about 5% w/w of at least one pH adjusting agent;
(e) about 10% to about 99.9% w/w of a pharmaceutically acceptable vehicle; and
(f) optionally at least one pharmaceutically acceptable excipient selected from complexing agents, binders (i.e., crystal growth inhibitors), and antioxidants;
wherein the composition is substantially buffer free.

In some embodiments, the present disclosure relates to an oral liquid pharmaceutical composition comprising fingolimod or a pharmaceutically acceptable salt thereof and at least one pharmaceutically acceptable excipient selected from preservatives, sweetening agents, pH adjusting agents such as an alkalizer, crystal growth inhibitors, and antioxidants; wherein the composition is substantially free of complexing agents.

In an embodiment, the present disclosure relates to an oral liquid pharmaceutical composition comprising:
(a) about 0.001% to about 0.1% w/w of fingolimod or a pharmaceutically acceptable salt thereof;
(b) about 0.01% to about 3% w/w of at least one preservative;
(c) optionally, about 0.01% to about 3% w/w of at least one sweetening agent;
(d) about 0.1% to about 5% w/w of at least one pH adjusting agent;
(e) about 10% to about 99.9% w/w of a pharmaceutically acceptable vehicle; and
(f) optionally at least one pharmaceutically acceptable excipient selected from buffering agents, binders (i.e., crystal growth inhibitors), and antioxidants;
wherein the composition is substantially free of complexing agent.

In some embodiments, the present disclosure relates to an oral liquid pharmaceutical composition comprising fingolimod or a pharmaceutically acceptable salt thereof and at least one pharmaceutically acceptable excipient selected from a preservative, sweetening agent, pH adjusting agent such as an alkalizer, crystal growth inhibitor, and antioxidant; wherein the composition is substantially free of cyclodextrin or derivatives thereof.

In an embodiment, the present disclosure relates to an oral liquid pharmaceutical composition comprising:
(a) about 0.001% to about 0.1% w/w of fingolimod or a pharmaceutically acceptable salt thereof;
(b) about 0.01% to about 3% w/w of at least one preservative;
(c) optionally, about 0.01% to about 3% w/w of at least one sweetening agent;
(d) about 0.1% to about 5% w/w of at least one pH adjusting agent;
(e) about 10% to about 99.9% w/w of a pharmaceutically acceptable vehicle; and
(f) optionally at least one pharmaceutically acceptable excipient selected from buffering agents, binders (i.e., crystal growth inhibitors), and antioxidants;
wherein the composition is substantially free of cyclodextrin or derivatives thereof.

In an embodiment, the present disclosure relates to an oral liquid pharmaceutical composition comprising:
(a) about 0.001% to about 0.1% w/w of fingolimod or a pharmaceutically acceptable salt thereof;
(b) about 0.01% to about 3% w/w of at least one preservative;
(c) optionally, about 0.01% to about 3% w/w of at least one sweetening agent;
(d) about 0.1% to about 5% w/w of at least one pH adjusting agent;
(e) about 10% to about 99.9% w/w of a pharmaceutically acceptable vehicle; and
(f) optionally at least one pharmaceutically acceptable excipient selected from buffering agents, binders (i.e., crystal growth inhibitors), and antioxidants;
wherein the composition is stable for at least 12 months at about 25° C. and about 60% RH.

In an embodiment, the present disclosure relates to an oral liquid pharmaceutical composition comprising:
(a) about 0.001% to about 0.1% w/w of fingolimod or a pharmaceutically acceptable salt thereof;
(b) about 0.01% to about 3% w/w of at least one preservative;
(c) optionally, about 0.01% to about 3% w/w of at least one sweetening agent;
(d) about 0.1% to about 5% w/w of at least one pH adjusting agent;
(e) about 10% to about 99.9% w/w of a pharmaceutically acceptable vehicle; and
(f) optionally at least one pharmaceutically acceptable excipient selected from buffering agents, binders (i.e., crystal growth inhibitors), and antioxidants;
   wherein the composition is stable for at least 6 months at about 25° C. and about 60% RH.

In an embodiment, the present disclosure relates to an oral liquid pharmaceutical composition comprising:
(a) about 0.001% to about 0.1% w/w of fingolimod or a pharmaceutically acceptable salt thereof;
(b) about 0.01% to about 3% w/w of at least one preservative;
(c) optionally, about 0.01% to about 3% w/w of at least one sweetening agent;
(d) about 0.1% to about 5% w/w of at least one pH adjusting agent;
(e) about 10% to about 99.9% w/w of a pharmaceutically acceptable vehicle; and
(f) optionally at least one pharmaceutically acceptable excipient selected from buffering agents, binders (i.e., crystal growth inhibitors), and antioxidants;
   wherein the composition is stable for at least 6 months at about 40° C. and about 75% RH.

In some embodiments, the present disclosure relates to an oral liquid pharmaceutical composition comprising fingolimod or a pharmaceutically acceptable salt thereof and a pharmaceutically acceptable vehicle, wherein the vehicle is not propylene glycol.

In some embodiments, the present disclosure relates to an oral liquid pharmaceutical composition comprising fingolimod or a salt thereof, wherein the composition is a stable aqueous solution.

In some embodiments, the present disclosure relates to an oral liquid composition pharmaceutical comprising fingolimod or a salt thereof, wherein the composition comprises at least one preservative and at least one pH adjuster.

In some embodiments, the present disclosure relates to an oral liquid pharmaceutical composition comprising fingolimod or a salt thereof, wherein the composition comprises at least one pH adjuster selected from HCl and NaOH.

In some embodiments, the present disclosure relates to an oral liquid pharmaceutical composition comprising fingolimod or a salt thereof, wherein the composition is stable after at least one freeze-thaw cycle.

In some embodiments, the present disclosure relates to an oral liquid pharmaceutical composition comprising fingolimod or a salt thereof, wherein the composition is photostable.

In some embodiments, the present disclosure relates to an oral liquid pharmaceutical composition consisting essentially of fingolimod or a salt thereof and at least one pharmaceutical acceptable vehicle.

In some embodiments, the present disclosure relates to an oral liquid pharmaceutical composition consisting essentially of fingolimod or a salt thereof, at least one pharmaceutical acceptable vehicle and at least one pH adjuster.

In some embodiments, the present disclosure relates to an oral liquid pharmaceutical composition consisting essentially of fingolimod or a salt thereof, at least one pharmaceutical acceptable vehicle and at least one preservative.

In some embodiments, the present disclosure relates to an oral liquid pharmaceutical composition consisting essentially of fingolimod or a salt thereof, at least one pharmaceutical acceptable vehicle, at least one pH adjuster, and at least one preservative.

In some embodiments, the oral liquid pharmaceutical composition comprises fingolimod and/or fingolimod hydrochloride. In some embodiments, the liquid composition comprises fingolimod. In some embodiments, the liquid composition comprises fingolimod hydrochloride.

In some embodiments, the present disclosure relates to an oral liquid pharmaceutical composition comprising fingolimod or a pharmaceutically acceptable salt thereof, wherein the concentration of fingolimod is about 0.1 mg/mL.

In some embodiments, the oral liquid pharmaceutical composition is an aqueous composition.

In some embodiments, the present disclosure relates to an oral liquid pharmaceutical composition comprising fingolimod or a pharmaceutically acceptable salt thereof, wherein the composition is stable at room temperature for at least 12 months.

In some embodiments, the present disclosure relates to an oral liquid pharmaceutical composition comprising fingolimod or a pharmaceutically acceptable salt thereof, wherein the pH of said composition is ranging from about 3 to about 6.

In some embodiments, the present disclosure relates to an oral liquid pharmaceutical composition comprising fingolimod or a pharmaceutically acceptable salt thereof, wherein the pH of said composition is about 4.5.

In some embodiments, the present disclosure relates to an oral liquid pharmaceutical composition comprising fingolimod or a pharmaceutically acceptable salt thereof, wherein the composition is ready to use and does not need further dilution.

In some embodiments, the oral liquid pharmaceutical composition is a solution. In some embodiments, the liquid composition is a clear solution. In some embodiments, the solution is clear and colorless. In some embodiments, the liquid composition is a clear solution under acidic conditions. In some embodiments, the liquid composition is free of precipitates. In some embodiments, the liquid composition is free of visible particulates. In some embodiments, the liquid composition is free of any crystals.

In some embodiments, the oral liquid pharmaceutical composition is ready to use (RTU). In some embodiments, the liquid composition is suitable for administration without diluting the composition prior to administration. In some embodiments, the liquid composition is shelf stable. In some embodiments, the liquid composition is suitable for oral delivery. In some embodiments, the liquid composition is in the form of an oral dosage form.

In some embodiments, the oral liquid pharmaceutical composition is palatable. In some embodiments, the composition is taste-masked. In some embodiments, the composition has a neutral and/or sweet taste. In some embodiments, the composition is not bitter.

In some embodiments, the oral liquid pharmaceutical composition comprising fingolimod or a salt thereof is easier to swallow than a solid composition comprising fingolimod or a salt thereof. In some embodiments, the liquid composition provides ease of dosing. In some embodiments, the liquid composition increases patient compliance due to, for example, ease of dosing and/or administration. In some embodiments, the liquid composition provides accurate dosing. In some embodiments, the liquid composition provides more accurate dosing compared to, for example, crushing a solid dosage comprising fingolimod or a salt thereof and mixing it with a liquid carrier. In some embodiments, the liquid composition comprises minimal ingredients in order to, for example, reduce the potential for reactions, such as allergic reactions, to the composition.

In some embodiments, the liquid composition provides an effective option for patients who have an aversion to or have trouble swallowing (e.g., dysphagia) solid dosage forms, particularly geriatric or pediatric patients.

### Excipients

In some embodiments, the oral liquid pharmaceutical compositions described herein comprise at least one excipient.

In some embodiments, the composition comprises at least one pH adjuster. In some embodiments, the pH adjuster is selected from HCl, NaOH, meglumine, and L-Arginine. In some embodiments, the pH adjuster is HCl or NaOH. In some embodiments, the pH adjuster is HCl. In some embodiments, the pH adjuster is NaOH. In some embodiments, the composition comprises HCl and NaOH.

In some embodiments, the composition pH adjuster is an alkalizer. In some embodiments, the total amount of alkalizer in the composition is from about 0.1% to about 0.5% w/w or from about 0.25% w/w to about 0.5% w/w. In some embodiments, the total amount of alkalizer in the composition is about 0.25% w/w. In some embodiments, the alkalizer is meglumine or L-Arginine.

In some embodiments, the composition comprises at least one preservative. In some embodiments, the composition comprises at least two preservatives. In some embodiments, the preservative or preservatives are selected from a paraben, sodium propionate (such as sodium propionate anhydrous), benzyl alcohol, benzalkonium chloride, benzyl benzoate, chlorobutanol, phenol, sodium benzoate, boric acid, benzoic acid, and formaldehyde. In some embodiments, the preservative or preservatives are selected from a paraben, sodium propionate, and benzyl alcohol.

In some embodiments, the composition comprises at least one paraben. In some embodiments, the composition comprises at least two parabens. In some embodiments, the composition comprises at least three parabens.

In some embodiments, the composition comprises methylparaben. In some embodiments, the composition comprises propylparaben. In some embodiments, the composition comprises methylparaben and propylparaben. In some embodiments, the composition comprises sodium propionate, such as sodium propionate anhydrous. In some embodiments, the composition comprises benzyl alcohol.

In some embodiments, the total amount of preservative is from about 0.001% to about 5.0% w/w. In some embodiments, the total amount of preservative is from about 0.001% to about 4.0% w/w. In some embodiments, the total amount of preservative is from about 0.001% to about 3.0% w/w. In some embodiments, the total amount of preservative is from about 0.001% to about 2.0% w/w. In some embodiments, the total amount of preservative is from about 0.001% to about 1.75% w/w. In some embodiments, the total amount of preservative is from about 0.001% to about 1.50% w/w. In some embodiments, the total amount of preservative is from about 0.001% to about 1.0% w/w. In some embodiments, the total amount of preservative is from about 0.001% to about 0.75% w/w. In some embodiments, the total amount of preservative is from about 0.001% to about 0.50% w/w. In some embodiments, the total amount of preservative is from about 0.001% to about 0.25% w/w. In some embodiments, the total amount of preservative is from about 0.001% to about 0.20% w/w. In some embodiments, the total amount of preservative is from about 0.001% to about 0.15% w/w. In some embodiments, the total amount of preservative is from about 0.001% to about 0.10% w/w.

In some embodiments, the total amount of preservative is from about 0.01% to about 2.50% w/w, from about 0.01% to about 2.00% w/w, from about 0.01% to about 1.75% w/w, from about 0.01% to about 1.50% w/w, from about 0.01% to about 1.25% w/w, from about 0.01% to about 1.00% w/w, from about 0.01% to about 0.75% w/w, from about 0.01% to about 0.50% w/w, from about 0.01% to about 0.25% w/w, from about 0.01% to about 0.20% w/w, from about 0.01% to about 0.15% w/w, or from about 0.01% to about 0.10% w/w.

In some embodiments, the total amount of preservative is from about 0.02% to about 2.50% w/w, from about 0.02% to about 2.00% w/w, from about 0.02% to about 1.75% w/w, from about 0.02% to about 1.50% w/w, from about 0.02% to about 1.25% w/w, from about 0.02% to about 1.00% w/w, from about 0.02% to about 0.75% w/w, from about 0.02% to about 0.50% w/w, from about 0.02% to about 0.25% w/w, from about 0.02% to about 0.20% w/w, from about 0.02% to about 0.15% w/w, or from about 0.02% to about 0.10% w/w.

In some embodiments, the total amount of preservative is from about 0.50% to about 2.50% w/w, from about 0.50% to about 2.00% w/w, from about 0.50% to about 1.75% w/w, from about 0.50% to about 1.50% w/w, from about 0.50% to about 1.25% w/w, from about 0.50% to about 1.00% w/w, or from about 0.50% to about 0.75% w/w.

In some embodiments, the total amount of preservative is from about 0.10% to about 5.0% w/w. In some embodiments, the total amount of preservative is from about 0.10% to about 4.0% w/w. In some embodiments, the total amount of preservative is from about 0.10% to about 3.0% w/w. In some embodiments, the total amount of preservative is from about 0.10% to about 2.0% w/w. In some embodiments, the total amount of preservative is from about 0.10% to about 1.75% w/w. In some embodiments, the total amount of preservative is from about 0.10% to about 1.50% w/w. In some embodiments, the total amount of preservative is from about 0.10% to about 1.0% w/w. In some embodiments, the total amount of preservative is from about 0.10% to about 0.75% w/w. In some embodiments, the total amount of preservative is from about 0.10% to about 0.50% w/w. In some embodiments, the total amount of preservative is from about 0.10% to about 0.25% w/w. In some embodiments, the total amount of preservative is from about 0.10% to about 0.20% w/w. In some embodiments, the total amount of preservative is from about 0.10% to about 0.15% w/w.

In some embodiments, the composition comprises at least one excipient selected from a preservative, a pH adjuster, a sweetener, a flavoring agent, a binder (i.e., crystal growth inhibitor), a buffering agent, a co-solvent, a surfactant, an antioxidant, a polymer, an anti-caking agent, an antifoaming agent, a diluent, a lubricant, a complexing agent, an anti-oxidant, a solubilizer, and a viscosity agent. In some embodiments, the composition comprises at least one excipient selected from a preservative, a pH adjuster, a sweetener, a flavoring agent, a binder, a buffering agent, a co-solvent, a surfactant, and a viscosity agent. In some embodiments, the composition comprises at least one excipient selected from a preservative, a pH adjuster, a sweetener, a flavoring agent, and a binder.

In some embodiments, the composition comprises at least one preservative and at least one pH adjuster. In some embodiments, the composition comprises at least two preservatives and at least one pH adjuster. In some embodiments, the composition comprises at least one preservative and at least two pH adjusters. In some embodiments, the composition comprises at least two preservatives and at least two pH adjusters. In some embodiments, the composition further comprises at least one preservative, at least one pH adjuster, and at least one binder.

In some embodiments, the composition optionally comprises at least one sweetener. In some embodiments, the total amount of sweetener in the composition is from about 0.005% to about 0.05% w/w. In some embodiments, the total amount of sweetener in the composition is from about 0.01% to about 0.025% w/w. In some embodiments, the sweetener is sucralose or xylitol.

In some embodiments, the composition comprises at least one flavoring agent. In some embodiments, the flavoring agent is grape flavor.

In some embodiments, the composition comprises at least one binder. In some embodiments, the total amount of the crystal growth inhibitor in the composition is from about 0.5% to about 1.5% w/w. In some embodiments, the total amount of the binder in the composition is about 1.0% w/w. In some embodiments, the binder is hypromellose or povidone.

In some embodiments, the total amount of excipient or excipients, other than a vehicle, in the composition is from about 0.005% to about 2.00% w/w.

In some embodiments, the total amount of excipient or excipients, other than a vehicle, is from about 0.01% to about 2.00% w/w, from about 0.01% to about 1.75% w/w, from about 0.01% to about 1.50% w/w, from about 0.01% to about 1.25% w/w, from about 0.01% to about 1.00% w/w, or from about 0.01% to about 0.75% w/w. In some embodiments, the total amount of excipient or excipients, other than a vehicle, is from about 0.01% to about 0.50% w/w. In some embodiments, the total amount of excipient or excipients, other than a vehicle, is from about 0.01% to about 0.25% w/w.

In some embodiments, the total amount of excipient or excipients, other than a vehicle, is from about 0.05% to about 2.00% w/w, from about 0.05% to about 1.75% w/w, from about 0.05% to about 1.50% w/w, from about 0.05% to about 1.25% w/w, from about 0.05% to about 1.00% w/w, or from about 0.05% to about 0.75% w/w. In some embodiments, the total amount of excipient or excipients, other than a vehicle, is from about 0.05% to about 0.50% w/w. In some embodiments, the total amount of excipient or excipients, other than a vehicle, is from about 0.05% to about 0.25% w/w.

In some embodiments, the total amount of excipient or excipients, other than a vehicle, is from about 0.10% to about 2.00% w/w, from about 0.10% to about 1.75% w/w, from about 0.10% to about 1.50% w/w, from about 0.10% to about 1.25% w/w, from about 0.10% to about 1.00% w/w, or from about 0.10% to about 0.75% w/w. In some embodiments, the total amount of excipient or excipients, other than a vehicle, is from about 0.10% to about 0.50% w/w.

In some embodiments, the total amount of excipient or excipients, other than a vehicle, is from about 0.10% to about 0.25% w/w.

In some embodiments, the liquid composition comprises a vehicle. In some embodiments, the vehicle comprises water. In some embodiments, the vehicle comprises water and another solvent.

In some embodiments, the vehicle comprises water and a solvent chosen from alcohol, glycerol, and propylene glycol. In some embodiments, the vehicle comprises water and a solvent chosen from alcohol and glycerol. In some embodiments, the liquid composition is an aqueous composition.

In some embodiments, the total percentage of the vehicle in the composition is from about 10.00% to 99.99% w/w. In some embodiments, the total percentage of the vehicle in the composition is from about 20.0% to about 99.9% w/w. In some embodiments, the total percentage of the vehicle in the composition is from about 30.0% to about 99.9% w/w. In some embodiments, the total percentage of the vehicle in the composition is from about 40.0% to about 99.9% w/w. In some embodiments, the total percentage of the vehicle in the composition is from about 50.0% to about 99.9% w/w. In some embodiments, the total percentage of the vehicle in the composition is from about 60.0% to about 99.9% w/w. In some embodiments, the total percentage of the vehicle in the composition is from about 70.0% to about 99.9% w/w. In some embodiments, the total percentage of the vehicle in the composition is from about 80.0% to about 99.9% w/w. In some embodiments, the total percentage of the vehicle in the composition is from about 85.0% to about 99.9% w/w, from about 90.0% to about 99.9% w/w, from about 95.0% to about 99.9%, from about 96.0% to about 99.9%, from about 97.0% to about 99.9%, from about 98.0% to about 99.9%, or from about 99.0% to about 99.9%.

In some embodiments, the total percentage of the vehicle in the composition is about 98.0%, about 98.1%, about 98.2%, about 98.3%, about 98.4%, about 98.5%, about 98.6%, about 98.7%, about 98.8%, or about 98.9%. In some embodiments, the total percentage of the vehicle in the composition is about 99.0%, about 99.1%, about 99.2%, about 99.3%, about 99.4%, about 99.5%, about 99.6%, about 99.7%, about 99.8%, or about 99.9%.

In some embodiments, the total volume of the vehicle in the composition is about 50 mL to about 1250 mL. In some embodiments, the total volume of the vehicle in the composition is about 100 mL to about 1250 mL, from about 150 mL to about 1250 mL, from about 200 mL to about 1250 mL, from about 250 mL to about 1250 mL, from about 300 mL to about 1250 mL, from about 400 mL to about 1250 mL, from about 500 mL to about 1250 mL, from about 600 mL to about 1250 mL, from about 700 mL to about 1250 mL, from about 800 mL to about 1250 mL, from about 900 mL to about 1250 mL, from about 1000 mL to about 1250 mL, from about 1100 mL to about 1250 mL, or from about 1200 mL to about 1250 mL.

In some embodiments, the total volume of the vehicle in the composition is about 50 mL to about 1000 mL, from about 100 mL to about 1000 mL, from about 150 mL to about 1000 mL, from about 200 mL to about 1000 mL, from about 250 mL to about 1000 mL, from about 300 mL to about 1000 mL, from about 400 mL to about 1000 mL, from about 500 mL to about 1000 mL, from about 600 mL to about 1000 mL, from about 700 mL to about 1000 mL, from about 800 mL to about 1000 mL, or from about 900 mL to about 1000 mL.

In some embodiments, the total volume of the vehicle in the composition is about 50 mL to about 500 mL, from about 100 mL to about 500 mL, from about 150 mL to about 500 mL, from about 200 mL to about 500 mL, from about 250 mL to about 500 mL, from about 300 mL to about 500 mL, or from about 400 mL to about 500 mL.

In some embodiments, the total volume of the vehicle in the composition is about 50 mL to about 200 mL, from about 100 mL to about 200 mL, or from about 150 mL to about 200 mL.

In some embodiments, the total volume of the vehicle in the composition is about 50 mL to about 150 mL or from about 100 mL to about 150 mL. In some embodiments, the total volume of the vehicle in the composition is about 50 mL to about 100 mL.

In some embodiments, the total volume of the vehicle in the composition is about 1250 mL. In some embodiments, the total volume of the vehicle in the composition is about 1000 mL. In some embodiments, the total volume of the vehicle in the composition is about 500 mL. In some embodiments, the total volume of the vehicle in the composition is about 250 mL. In some embodiments, the total volume of the vehicle in the composition is about 200 mL. In some embodiments, the total volume of the vehicle in the composition is about 100 mL. In some embodiments, the total volume of the vehicle in the composition is about 150 mL. In some embodiments, the total volume of the vehicle in the composition is about 50 mL.

In some embodiments, the composition does not comprise glycerin, propylene glycol, simethicone, cyclodextrin or a derivative thereof, and/or polysorbate 80. In some embodiments, the composition does not comprise glycerin. In some embodiments, the composition does not comprise propylene glycol. In some embodiments, the composition does not comprise simethicone. In some embodiments, the composition does not comprise polysorbate 80.

### Stability

In some embodiments, the composition disclosed herein is stable.

In some embodiments, the composition is stable in that no precipitation occurs when the composition is subjected to certain specified conditions. In some embodiments, the composition is stable in that no visible particulates form when the composition is subjected to certain specified conditions. In some embodiments, the composition is stable in that it is free of crystalline deposits when the composition is subjected to certain specified conditions.

In some embodiments, the composition is stable in that the fingolimod or salt thereof in the composition does not degrade, or degrades only slightly, when the composition is subjected to certain specified conditions (e.g., specific temperature, specific relative humidity, freeze-thaw, illumination/ultraviolet energy). For example, in some embodiments, the amount of fingolimod or salt thereof in the composition after being subject to certain specified conditions is about 75% to about 110%, from about 80% to about 110%, from about 85% to about 110%, from about 90% to about 110%, from about 95% to about 110%, from about 96% to about 110%, from about 97% to about 110%, from about 98% to about 110%, or from about 99% to about 110%. In some embodiments, the amount of fingolimod or salt thereof in the composition is measured using HPLC, for example, as described in the Examples.

In some embodiments, the composition is stable in that minimal impurities form when the composition is subjected to certain specified conditions (e.g., specific temperature, specific relative humidity, freeze-thaw, illumination/ultraviolet energy). In some embodiments, “minimal impurities” means not more than about 2% of total impurities in the composition, when measured using HPLC, for example, as described in the Examples. In some embodiments, the total amount of impurities in the compositions disclosed herein is not more than about 5%. In some embodiments, the total amount of impurities in the composition is not more than about 4%, not more than about 3%, not more than about 2%, or not more than about 1%.

In some embodiments, “minimal impurities” may comprise at least one fingolimod related impurity (e.g., Impurity A or 2-Amino-2-(4-hexylphenethyl)propane-1,3-diol). In some embodiments, “minimal impurities” means not more than about 1% of a particular impurity in the composition, when measured using HPLC, for example, as described in the Examples. In some embodiments, the total amount of fingolimod related Impurity A in the compositions disclosed herein is not more than about 5%, not more than about 4%, not more than about 3%, not more than about 2%, not more than about 1%, or not more than 0.5%.

In some embodiments, the composition is stable upon preparation. In some embodiments, the composition is stable after storage.

In some embodiments, the composition is stable after storage in a closed container for about 1 month at about 40° C. and about 75% relative humidity. In some embodiments, the composition is stable after storage in a closed container for about 3 months at about 40° C. and about 75% relative humidity. In some embodiments, the composition is stable after storage in a closed container for about 6 months at about 40° C. and about 75% relative humidity. In some embodiments, the composition is stable after storage in a closed container for about 12 months at about 40° C. and about 75% relative humidity.

In some embodiments, the composition is stable after storage in a closed container for about 1 month at about 25° C. and about 60% relative humidity. In some embodiments, the composition is stable after storage in a closed container for about 3 months at about 25° C. and about 60% relative humidity. In some embodiments, the composition is stable after storage in a closed container for about 6 months at about 25° C. and about 60% relative humidity. In some embodiments, the composition is stable after storage in a closed container for about 12 months at about 25° C. and about 60% relative humidity.

In some embodiments, the composition is stable after storage in a closed container for about 1 month at about 60° C. In some embodiments, the composition is stable after storage in a closed container for about 3 months at about 60° C. In some embodiments, the composition is stable after storage in a closed container for about 6 months at about 60° C. In some embodiments, the composition is stable after storage in a closed container for about 12 months at about 60° C.

In some embodiments, the amount of fingolimod or a salt thereof in the compositions disclosed herein after storage is at least about 90%, when measured using HPLC, for example, as described in the Examples. In some embodiments, the amount of fingolimod or a salt thereof in the composition after storage is at least about 91%, at least about 92%, at least about 93%, at least about 94%, at least about 95%, at least about 96%, at least about 97%, at least about 98%, or at least about 99%. In some embodiments, the amount of fingolimod or a salt thereof in the composition after storage is at least about 91% to about 110%, from about 92% to about 110%, from about 93% to about 110%, from about 94% to about 110%, from about 95% to about 110%, from about 96% to about 110%, from about 97% to about 110%, from about 98% to about 110%, or from 99% to about 110%. In some embodiments, the amount of fingolimod or a salt thereof in the composition after storage is at least about 91% to about 105%, from about 92% to about 105%, from about 93% to about 105%, from about 94% to about 105%, from about 95% to about 105%, from about 96% to about 105%, from about 97% to about 105%, from about 98% to about 105%, or from 99% to about 105%.

In some embodiments, the total amount of impurities present after storage is not more than about 5%, when measured using HPLC, for example, as described in the Examples. In some embodiments, the total amount of impurities present after storage is not more than about 4%, not more than about 3%, not more than about 2%, or not more than about 1%. In some embodiments, the total amount of fingolimod related Impurity A present after storage is not more than about 5%, not more than about 4%, not more than about 3%, not more than about 2%, not more than about 1%, or not more than about 0.5%.

In some embodiments, the composition is stable after at least one freeze-thaw cycle. In some embodiments, the composition is stable after at least two freeze-thaw cycles. In some embodiments, the composition is stable after at least three freeze-thaw cycles. In some embodiments, the composition is stable after at least four freeze-thaw cycles. In some embodiments, the composition is stable after at least five freeze-thaw cycles. In some embodiments, each freeze-thaw cycle comprises about 48 hours at about −20° C., followed by about 48 hours at room temperature.

In some embodiments, the total amount of impurities present in the composition after at least one freeze-thaw cycle is not more than about 4%, not more than about 3%, not more than about 2%, or not more than about 1%. In some embodiments, the total amount of fingolimod related Impurity A present after at least one freeze-thaw cycle is not more than about 5%, not more than about 4%, not more than about 3%, not more than about 2%, not more than about 1%, or not more than 0.5%. In some embodiments, the amount of fingolimod or salt thereof is measured using HPLC, for example, as described in the Examples.

In some embodiments, the amount of fingolimod or salt thereof in the composition after at least one freeze-thaw cycle is from about 10% to about 110%, when measured using HPLC, for example, as described in the Examples. In some embodiments, the amount of fingolimod or salt thereof in the composition after at least one freeze-thaw cycle is from about 20% to about 110%, from about 30% to about 110%, from about 40% to about 110%, or from about 50% to about 110%. In some embodiments, the amount of fingolimod or salt thereof in the composition after at least one freeze-thaw cycle is from about 60% to about 110%. In some embodiments, the amount of fingolimod that or salt thereof in the composition at least one freeze-thaw cycle is from about 70% to about 110%. In some embodiments, the amount of fingolimod or salt thereof in the composition after at least one freeze-thaw cycle is from about 80% to about 110%. In some embodiments, the amount of fingolimod or salt thereof in the composition after at least one freeze-thaw cycle is from about 90% to about 110%. In some embodiments, the amount of fingolimod or salt thereof in the composition after at least one freeze-thaw cycle is from about 95% to about 110%. In some embodiments, the amount of fingolimod or salt thereof in the composition after at least one freeze-thaw cycle is from about 99% to about 110%.

In some embodiments, the composition is photostable. In some embodiments, the composition is photostable when exposed to an overall illumination of about 1.2 million lux hours. In some embodiments, the composition is photostable when exposed to an integrated near ultraviolet energy of about 200-watt hours/square meter.

In some embodiments, the total amount of impurities present in the composition after exposure to illumination or an integrated near ultraviolet energy is not more than about 4%, not more than about 3%, not more than about 2%, or not more than about 1%, when measured using HPLC, for example, as described in the Examples. In some embodiments, the total amount of fingolimod related Impurity A present after exposure to illumination or an integrated near ultraviolet energy is not more than about 5%, not more than about 4%, not more than about 3%, not more than about 2%, not more than about 1%, or not more than 0.5%. In some embodiments, the composition is photostable when packaged in a container(s), such as a bottle or a box (such as a monocarton box).

In some embodiments, the amount of fingolimod or salt thereof in the composition after exposure to illumination or an integrated near ultraviolet energy is from about 10% to about 110%, from about 20% to about 110%, from about 30% to about 110%, from about 40% to about 110%, from about 50% to about 110%, from about 60% to about 110%, from about 70% to about 110%, from about 80% to about 110%, or from about 90% to about 110%, when measured using HPLC, for example, as described in the Examples. In some embodiments, the amount of fingolimod or salt thereof in the composition after exposure to illumination or an integrated near ultraviolet energy is from about 95% to about 110%, from about 96% to about 110%, from about 97% to about 110%, from about 98% to about 110%, or from about 99% to about 110%. In some embodiments, the amount of fingolimod or salt thereof in the composition after exposure to illumination or an integrated near ultraviolet energy is about 95%, about 96%, about 97%, about 98%, about 99%, or about 100%.

In some embodiments, the oral liquid composition is shelf stable.

pH

In some embodiments, the composition has a pH of about 2.0 to about 9.0. In some embodiments, the composition has a pH of about 3.0 to about 9.0. In some embodiments, the composition has a pH of about 3.5 to about 9.0. In some embodiments, the composition has a pH of about 4.0 to about 9.0. In some embodiments, the composition has a pH of about 4.5 to about 9.0. In some embodiments, the composition has a pH of about 5.0 to about 9.0. In some embodiments, the composition has a pH of about 5.5 to about 9.0. In some embodiments, the composition has a pH of about 6.0 to about 9.0. In some embodiments, the composition has a pH of about 6.5 to about 9.0. In some embodiments, the composition has a pH of about 7.0 to about 9.0. In some embodiments, the composition has a pH of about 7.5 to about 9.0. In some embodiments, the composition has a pH of about 8.0 to about 9.0. In some embodiments, the composition has a pH of about 8.5 to about 9.0.

In some embodiments, the composition has a pH of about 2.5 to about 8.5, of about 3.5 to about 8.5, of about 4.5 to about 8.5, of about 5.5 to about 8.5, of about 6.5 to about 8.5, or of about 7.5 to about 8.5. In some embodiments, the composition has a pH of about 2.5 to about 7.5, of about 3.5 to about 7.5, of about 4.5 to about 7.5, of about 5.5 to about 7.5, or of about 6.5 to about 7.5.

In some embodiments, the composition has a pH of about 2.5 to about 6.5, of about 3.5 to about 6.5, of about 4.5 to about 6.5, or of about 5.5 to about 6.5. In some embodiments, the composition has a pH of about 2.5 to about 5.5, of about 3.5 to about 5.5, or of about 4.5 to about 5.5. In some embodiments, the composition has a pH of about 2.5 to about 4.5 or of about 3.5 to about 4.5.

In some embodiments, the composition has a pH of about 2.0 to about 8.0, of about 3.0 to about 8.0, of about 4.0 to about 8.0, of about 5.0 to about 8.0, of about 6.0 to about 8.0, or of about 7.0 to about 8.0.

In some embodiments, the composition has a pH of about 2.0 to about 7., of about 3.0 to about 7.0, of about 4.0 to about 7.0, of about 5.0 to about 7.0, or of about 6.0 to about 7.0.

In some embodiments, the composition has a pH of about 2.0 to about 6.0, of about 3.0 to about 6.0, of about 4.0 to about 6.0, or of about 5.0 to about 6.0.

In some embodiments, the composition has a pH of about 2.0 to about 5.0, or of about 3.0 to about 5.0, or of about 4.0 to about 5.0.

In some embodiments, the composition has a pH of about 2.0. In some embodiments, the composition has a pH of about 2.5. In some embodiments, the composition has a pH of about 3.0. In some embodiments, the composition has a pH of about 3.5. In some embodiments, the composition has a pH of about 4.0. In some embodiments, the composition has a pH of about 4.5. In some embodiments, the composition has a pH of about 5.0. In some embodiments, the composition has a pH of about 5.5. In some embodiments, the composition has a pH of about 6.0. In some embodiments, the composition has a pH of about 6.5. In some embodiments, the composition has a pH of about 7.0. In some embodiments, the composition has a pH of about 7.5. In some embodiments, the composition has a pH of about 8.0. In some embodiments, the composition has a pH of about 8.5. In some embodiments, the composition has a pH of about 9.0.

### Strength

In some embodiments, the oral liquid pharmaceutical compositions described herein comprise fingolimod or a pharmaceutically acceptable salt thereof at a concentration of about 0.01 mg/mL to about 10 mg/mL.

In some embodiments, the composition comprises an amount of fingolimod or salt thereof that is equivalent to from about 0.01 mg/mL to about 0.50 mg/mL of fingolimod (free base).

In some embodiments, the composition comprises an amount of fingolimod or salt thereof that is equivalent to from about 0.02 mg/mL to about 0.50 mg/mL, from about 0.03 mg/mL to about 0.50 mg/mL, from about 0.04 mg/mL to about 0.50 mg/mL, from about 0.05 mg/mL to about 0.50 mg/mL, from about 0.06 mg/mL to about 0.50 mg/mL, from about 0.07 mg/mL to about 0.50 mg/mL, from about 0.08 mg/mL to about 0.50 mg/mL, from about 0.09 mg/mL to about 0.50 mg/mL of fingolimod. In some embodiments, the composition comprises an amount of fingolimod or salt thereof that is equivalent to from about 0.10 mg/mL to about 0.50 mg/mL of fingolimod.

In some embodiments, the composition comprises an amount of fingolimod or a salt thereof that is equivalent to from about 0.11 mg/mL to about 0.50 mg/mL, from about 0.12 mg/mL to about 0.50 mg/mL, from about 0.13 mg/mL to about 0.50 mg/mL, from about 0.14 mg/mL to about 0.50 mg/mL, from about 0.15 mg/mL to about 0.50 mg/mL, from about 0.16 mg/mL to about 0.50 mg/mL, from about 0.17 mg/mL to about 0.50 mg/mL, from about 0.18 mg/mL to about 0.50 mg/mL, or from about 0.19 mg/mL to about 0.50 mg/mL of fingolimod.

In some embodiments, the composition comprises an amount of fingolimod or a salt thereof that is equivalent to from about 0.20 mg/mL to about 0.50 mg/mL of fingolimod. In some embodiments, the composition comprises an amount of fingolimod or a salt thereof that is equivalent to from about 0.30 mg/mL to about 0.50 mg/mL of fingolimod. In some embodiments, the composition comprises an amount of fingolimod or a salt thereof that is equivalent to from about 0.40 mg/mL to about 0.50 mg/mL of fingolimod.

In some embodiments, the composition comprises an amount of fingolimod or salt thereof that is equivalent to from about 0.01 mg/mL to about 0.40 mg/mL, from about 0.02 mg/mL to about 0.40 mg/mL, from about 0.03 mg/mL to about 0.40 mg/mL, from about 0.04 mg/mL to about 0.40 mg/mL, from about 0.05 mg/mL to about 0.40 mg/mL, from about 0.06 mg/mL to about 0.40 mg/mL, from about 0.07 mg/mL to about 0.40 mg/mL, from about 0.08 mg/mL to about 0.40 mg/mL, or from about 0.09 mg/mL to about 0.40 mg/mL of fingolimod. In some embodiments, the composition comprises an amount of fingolimod or a salt thereof that is equivalent to from about 0.10 mg/mL to about 0.40 mg/mL of fingolimod.

In some embodiments, the composition comprises an amount of fingolimod or a salt thereof that is equivalent to from about 0.11 mg/mL to about 0.40 mg/mL, from about 0.12 mg/mL to about 0.40 mg/mL, from about 0.13 mg/mL to about 0.40 mg/mL, from about 0.14 mg/mL to about 0.40 mg/mL, from about 0.15 mg/mL to about 0.40 mg/mL, from about 0.16 mg/mL to about 0.40 mg/mL, from about 0.17 mg/mL to about 0.40 mg/mL, from about 0.18 mg/mL to about 0.40 mg/mL, or from about 0.19 mg/mL to about 0.40 mg/mL of fingolimod.

In some embodiments, the composition comprises an amount of fingolimod or a salt thereof that is equivalent to from about 0.20 mg/mL to about 0.40 mg/mL of fingolimod. In some embodiments, the composition comprises an amount of fingolimod or a salt thereof that is equivalent to from about 0.30 mg/mL to about 0.40 mg/mL of fingolimod.

In some embodiments, the composition comprises an amount of fingolimod or salt thereof that is equivalent to from about 0.01 mg/mL to about 0.30 mg/mL, from about 0.02 mg/mL to about 0.30 mg/mL, from about 0.03 mg/mL to about 0.30 mg/mL, from about 0.04 mg/mL to about 0.30 mg/mL, from about 0.05 mg/mL to about 0.30 mg/mL, from about 0.06 mg/mL to about 0.30 mg/mL, from about 0.07 mg/mL to about 0.30 mg/mL, from about 0.08 mg/mL to about 0.30 mg/mL, or from about 0.09 mg/mL to about 0.30 mg/mL of fingolimod. In some embodiments, the composition comprises an amount of fingolimod or a salt thereof that is equivalent to from about 0.10 mg/mL to about 0.30 mg/mL of fingolimod.

In some embodiments, the composition comprises an amount of fingolimod or a salt thereof that is equivalent to from about 0.11 mg/mL to about 0.30 mg/mL, from about 0.12 mg/mL to about 0.30 mg/mL, from about 0.13 mg/mL to about 0.30 mg/mL, from about 0.14 mg/mL to about 0.30 mg/mL, from about 0.15 mg/mL to about 0.30 mg/mL, from about 0.16 mg/mL to about 0.30 mg/mL, from about 0.17 mg/mL to about 0.30 mg/mL, from about 0.18 mg/mL to about 0.30 mg/mL, from about 0.19 mg/mL to about 0.30 mg/mL, or from about 0.20 mg/mL to about 0.30 mg/mL of fingolimod.

In some embodiments, the composition comprises an amount of fingolimod or salt thereof that is equivalent to from about 0.01 mg/mL to about 0.20 mg/mL, from about 0.02 mg/mL to about 0.20 mg/mL, from about 0.03 mg/mL to about 0.20 mg/mL, from about 0.04 mg/mL to about 0.20 mg/mL, from about 0.05 mg/mL to about 0.20 mg/mL, from about 0.06 mg/mL to about 0.20 mg/mL, from about 0.07 mg/mL to about 0.20 mg/mL, from about 0.08 mg/mL to about 0.20 mg/mL, or from about 0.09 mg/mL to about 0.20 mg/mL of fingolimod. In some embodiments, the composition comprises an amount of fingolimod or a salt thereof that is equivalent to from about 0.10 mg/mL to about 0.20 mg/mL of fingolimod.

In some embodiments, the composition comprises an amount of fingolimod or a salt thereof that is equivalent to from about 0.11 mg/mL to about 0.20 mg/mL, from about 0.12 mg/mL to about 0.20 mg/mL, from about 0.13 mg/mL to about 0.20 mg/mL, from about 0.14 mg/mL to about 0.20 mg/mL, from about 0.15 mg/mL to about 0.20 mg/mL, from about 0.16 mg/mL to about 0.20 mg/mL, from about 0.17 mg/mL to about 0.20 mg/mL, from about 0.18 mg/mL to about 0.20 mg/mL, or from about 0.19 mg/mL to about 0.20 mg/mL of fingolimod.

In some embodiments, the composition comprises an amount of fingolimod or a salt thereof that is equivalent to about 0.01 mg/mL, about 0.02 mg/mL, about 0.03 mg/mL, about 0.04 mg/mL, about 0.05 mg/mL, about 0.06 mg/mL, about 0.07 mg/mL, about 0.08 mg/mL, or about 0.09 mg/mL of fingolimod. In some embodiments, the composition comprises an amount of fingolimod or a salt thereof that is equivalent to about 0.10 mg/mL of fingolimod.

In some embodiments, the composition comprises an amount of fingolimod or a salt thereof that is equivalent to about 0.11 mg/mL, about 0.12 mg/mL, about 0.13 mg/mL, about 0.14 mg/mL, about 0.15 mg/mL, about 0.16 mg/mL, about 0.17 mg/mL, about 0.18 mg/mL, about 0.19 mg/mL, or about 0.20 mg/mL of fingolimod.

In some embodiments, the composition comprises an amount of fingolimod or a salt thereof that is equivalent to about 0.21 mg/mL, about 0.22 mg/mL, about 0.23 mg/mL, about 0.24 mg/mL, about 0.25 mg/mL, about 0.26 mg/mL, about 0.27 mg/mL, about 0.28 mg/mL, about 0.29 mg/mL, or about 0.30 mg/mL of fingolimod.

In some embodiments, the composition comprises an amount of fingolimod or a salt thereof that is equivalent to about 0.31 mg/mL, about 0.32 mg/mL, about 0.33 mg/mL, about 0.34 mg/mL, about 0.35 mg/mL, about 0.36 mg/mL, about 0.37 mg/mL, about 0.38 mg/mL, about 0.39 mg/mL, or about 0.40 mg/mL of fingolimod.

In some embodiments, the composition comprises an amount of fingolimod or a salt thereof that is equivalent to about 0.41 mg/mL, about 0.42 mg/mL, about 0.43 mg/mL, about 0.44 mg/mL, about 0.45 mg/mL, about 0.46 mg/mL, about 0.47 mg/mL, about 0.48 mg/mL, about 0.49 mg/mL, or about 0.50 mg/mL of fingolimod.

### Kits and Packaged Products

In some embodiments, the present disclosure relates to a kit comprising:
(i) a first container comprising a liquid composition as described herein; and
(ii) a second container comprising a pharmaceutically acceptable vehicle.

In some embodiments, the present disclosure relates to a kit comprising:
(i) a first container comprising fingolimod or a salt thereof; and
(ii) a second container comprising a pharmaceutically acceptable vehicle.

In some embodiments, the first container comprises fingolimod or a salt thereof. In some embodiments, the fingolimod or a salt thereof is in the form of a powder. In some embodiments, the first container comprises fingolimod hydrochloride. In some embodiments, the fingolimod hydrochloride is in the form of a powder. In some embodiments, the first container comprises fingolimod and/or fingolimod hydrochloride. In some embodiments, the first container comprises fingolimod and/or fingolimod hydrochloride in the form of a powder.

In some embodiments, the first container further comprises a preservative.

In some embodiments, the total amount of preservative in the first container is from about 0.001% to about 5.0% w/w. In some embodiments, the total amount of preservative is from about 0.001% to about 4.0% w/w. In some embodiments, the total amount of preservative is from about 0.001% to about 3.0% w/w. In some embodiments, the total amount of preservative is from about 0.001% to about 2.0% w/w. In some embodiments, the total amount of preservative is from about 0.001% to about 1.75% w/w. In some embodiments, the total amount of preservative is from about 0.001% to about 1.50% w/w. In some embodiments, the total amount of preservative is from about 0.001% to about 1.0% w/w. In some embodiments, the total amount of preservative is from about 0.001% to about 0.75% w/w. In some embodiments, the total amount of preservative is from about 0.001% to about 0.50% w/w. In some embodiments, the total amount of preservative is from about 0.001% to about 0.25% w/w. In some embodiments, the total amount of preservative is from about 0.001% to about 0.20% w/w. In some embodiments, the total amount of preservative is from about 0.001% to about 0.15% w/w. In some embodiments, the total amount of preservative is from about 0.001% to about 0.10% w/w.

In some embodiments, the total amount of preservative is from about 0.01% to about 2.50% w/w, from about 0.01% to about 2.00% w/w, from about 0.01% to about 1.75% w/w, from about 0.01% to about 1.50% w/w, from about 0.01% to about 1.25% w/w, from about 0.01% to about 1.00% w/w, from about 0.01% to about 0.75% w/w, from about 0.01% to about 0.50% w/w, from about 0.01% to about 0.25% w/w, from about 0.01% to about 0.20% w/w, from about 0.01% to about 0.15% w/w, or from about 0.01% to about 0.10% w/w.

In some embodiments, the total amount of preservative is from about 0.02% to about 2.50% w/w, from about 0.02% to about 2.00% w/w, from about 0.02% to about 1.75% w/w, from about 0.02% to about 1.50% w/w, from about 0.02% to about 1.25% w/w, from about 0.02% to about 1.00% w/w, from about 0.02% to about 0.75% w/w, from about 0.02% to about 0.50% w/w, from about 0.02% to about 0.25% w/w, from about 0.02% to about 0.20% w/w, from about 0.02% to about 0.15% w/w, or from about 0.02% to about 0.10% w/w.

In some embodiments, the total amount of preservative is from about 0.50% to about 2.50% w/w, from about 0.50% to about 2.00% w/w, from about 0.50% to about 1.75% w/w, from about 0.50% to about 1.50% w/w, from about 0.50% to about 1.25% w/w, from about 0.50% to about 1.00% w/w, or from about 0.50% to about 0.75% w/w.

In some embodiments, the total amount of preservative is from about 0.10% to about 5.0% w/w. In some embodiments, the total amount of preservative is from about 0.10% to about 4.0% w/w. In some embodiments, the total amount of preservative is from about 0.10% to about 3.0% w/w. In some embodiments, the total amount of preservative is from about 0.10% to about 2.0% w/w. In some embodiments, the total amount of preservative is from about 0.10% to about 1.75% w/w. In some embodiments, the total amount of preservative is from about 0.10% to about 1.50% w/w. In some embodiments, the total amount of preservative is from about 0.10% to about 1.0% w/w. In some embodiments, the total amount of preservative is from about 0.10% to about 0.75% w/w. In some embodiments, the total amount of preservative is from about 0.10% to about 0.50% w/w. In some embodiments, the total amount of preservative is from about 0.10% to about 0.25% w/w. In some embodiments, the total amount of preservative is from about 0.10% to about 0.20% w/w. In some embodiments, the total amount of preservative is from about 0.10% to about 0.15% w/w.

In some embodiments, the first container further comprises at least one excipient selected from a preservative, a pH adjuster, a sweetener, a flavoring agent, a binder (i.e., a crystal growth inhibitor), a buffering agent, a co-solvent, a surfactant, an antioxidant, a polymer, an anti-caking agent, an antifoaming agent, a diluent, a lubricant, and a viscosity agent. In some embodiments, the first container further comprises at least one excipient selected from a pH adjuster, a sweetener, a flavoring agent, a binder, a buffering agent, an anti-caking agent, an antifoaming agent, a diluent, and a lubricant. In some embodiments, the first container optionally further comprises at least one sweetener. In some embodiments, the first container further comprises at least one flavoring agent. In some embodiments, the first container further comprises at least one binder. In some embodiments, the first container further comprises at least one pH adjuster.

In some embodiments, the total amount of excipient or excipients in the first container, other than a vehicle, is from about 0.005% to about 2.00% w/w.

In some embodiments, the total amount of excipient or excipients in the first container, other than a vehicle, is from about 0.01% to about 2.00% w/w, from about 0.01% to about 1.75% w/w, from about 0.01% to about 1.50% w/w, from about 0.01% to about 1.25% w/w, from about 0.01% to about 1.00% w/w, or from about 0.01% to about 0.75% w/w. In some embodiments, the total amount of excipient or excipients, other than a vehicle, is from about 0.01% to about 0.50% w/w. In some embodiments, the total amount of excipient or excipients, other than a vehicle, is from about 0.01% to about 0.25% w/w.

In some embodiments, the total amount of excipient or excipients in the first container, other than a vehicle, is from about 0.05% to about 2.00% w/w, from about 0.05% to about 1.75% w/w, from about 0.05% to about 1.50% w/w, from about 0.05% to about 1.25% w/w, from about 0.05% to about 1.00% w/w, or from about 0.05% to about 0.75% w/w. In some embodiments, the total amount of excipient or excipients, other than a vehicle, is from about 0.05% to about 0.50% w/w. In some embodiments, the total amount of excipient or excipients, other than a vehicle, is from about 0.05% to about 0.25% w/w.

In some embodiments, the total amount of excipient or excipients in the first container, other than a vehicle, is from about 0.10% to about 2.00% w/w, from about 0.10% to about 1.75% w/w, from about 0.10% to about 1.50% w/w, from about 0.10% to about 1.25% w/w, from about 0.10% to about 1.00% w/w, or from about 0.10% to about 0.75% w/w. In some embodiments, the total amount of excipient or excipients, other than a vehicle, is from about 0.10% to about 0.50% w/w. In some embodiments, the total amount of excipient or excipients, other than a vehicle, is from about 0.10% to about 0.25% w/w.

In some embodiments, the vehicle in the second container comprises water. In some embodiments, the vehicle comprises water. In some embodiments, the vehicle comprises water and another solvent. In some embodiments, the vehicle comprises water and a solvent chosen from alcohol, glycerol, and propylene glycol. In some embodiments, the vehicle comprises water and a solvent chosen from alcohol and glycerol.

In some embodiments, the second container comprises from about 50 mL to about 1250 mL of the vehicle. In some embodiments, the second container comprises from about 100 mL to about 1250 mL of the vehicle, from about 150 mL to about 1250 mL of the vehicle, from about 200 mL to about 1250 mL of the vehicle, from about 250 mL to about 1250 mL of the vehicle, from about 300 mL to about 1250 mL of the vehicle, from about 400 mL to about 1250 mL of the vehicle, from about 500 mL to about 1250 mL of the vehicle, from about 600 mL to about 1250 mL of the vehicle, from about 700 mL to about 1250 mL of the vehicle, from about 800 mL to about 1250 mL of the vehicle, from about 900 mL to about 1250 mL of the vehicle, from about 1000 mL to about 1250 mL of the vehicle, from about 1100 mL to about 1250 mL of the vehicle, or from about 1200 mL to about 1250 mL of the vehicle.

In some embodiments, the second container comprises from about 50 mL to about 1000 mL of the vehicle, from about 100 mL to about 1000 mL of the vehicle, from about 150 mL to about 1000 mL of the vehicle, from about 200 mL to about 1000 mL of the vehicle, from about 250 mL to about 1000 mL of the vehicle, from about 300 mL to about 1000 mL of the vehicle, from about 400 mL to about 1000 mL of the vehicle, from about 500 mL to about 1000 mL of the vehicle, from about 600 mL to about 1000 mL of the vehicle, from about 700 mL to about 1000 mL of the vehicle, from about 800 mL to about 1000 mL of the vehicle, or from about 900 mL to about 1000 mL of the vehicle.

In some embodiments, the second container comprises from about 50 mL to about 500 mL of the vehicle, from about 100 mL to about 500 mL of the vehicle, from about 150 mL to about 500 mL of the vehicle, from about 200 mL to about 500 mL of the vehicle, from about 250 mL to about 500 mL of the vehicle, from about 300 mL to about 500 mL of the vehicle, or from about 400 mL to about 500 mL of the vehicle.

In some embodiments, the second container comprises from about 50 mL to about 200 mL of the vehicle, from about 100 mL to about 200 mL of the vehicle, or from about 150 mL to about 200 mL of the vehicle.

In some embodiments, the second container comprises from about 50 mL to about 150 mL of the vehicle or from about 100 mL to about 150 mL of the vehicle. In some embodiments, the second container comprises from about 50 mL to about 100 mL of the vehicle.

In some embodiments, the second container comprises about 1250 mL of the vehicle. In some embodiments, the second container comprises about 1000 mL of the vehicle. In some embodiments, the second container comprises about 500 mL of the vehicle. In some embodiments, the second container comprises about 250 mL of the vehicle. In some embodiments, the second container comprises about 200 mL of the vehicle. In some embodiments, the second container comprises about 100 mL of the vehicle. In some embodiments, the second container comprises about 150 mL of the vehicle. In some embodiments, the second container comprises about 50 mL of the vehicle.

In some embodiments, the vehicle in the second container further comprises a preservative.

In some embodiments, the total amount of preservative in the vehicle is from about 0.001% to about 5.0% w/w. In some embodiments, the total amount of preservative is from about 0.001% to about 4.0% w/w. In some embodiments, the total amount of preservative is from about 0.001% to about 3.0% w/w. In some embodiments, the total amount of preservative is from about 0.001% to about 2.0% w/w. In some embodiments, the total amount of preservative is from about 0.001% to about 1.75% w/w. In some embodiments, the total amount of preservative is from about 0.001% to about 1.50% w/w. In some embodiments, the total amount of preservative is from about 0.001% to about 1.0% w/w. In some embodiments, the total amount of preservative is from about 0.001% to about 0.75% w/w. In some embodiments, the total amount of preservative is from about 0.001% to about 0.50% w/w. In some embodiments, the total amount of preservative is from about 0.001% to about 0.25% w/w. In some embodiments, the total amount of preservative is from about 0.001% to about 0.20% w/w. In some embodiments, the total amount of preservative is from about 0.001% to about 0.15% w/w. In some embodiments, the total amount of preservative is from about 0.001% to about 0.10% w/w.

In some embodiments, the total amount of preservative is from about 0.01% to about 2.50% w/w, from about 0.01% to about 2.00% w/w, from about 0.01% to about 1.75% w/w, from about 0.01% to about 1.50% w/w, from about 0.01% to about 1.25% w/w, from about 0.01% to about 1.00% w/w, from about 0.01% to about 0.75% w/w, from about 0.01% to about 0.50% w/w, from about 0.01% to about 0.25% w/w, from about 0.01% to about 0.20% w/w, from about 0.01% to about 0.15% w/w, or from about 0.01% to about 0.10% w/w.

In some embodiments, the total amount of preservative is from about 0.02% to about 2.50% w/w, from about 0.02% to about 2.00% w/w, from about 0.02% to about 1.75% w/w, from about 0.02% to about 1.50% w/w, from about 0.02% to about 1.25% w/w, from about 0.02% to about 1.00% w/w, from about 0.02% to about 0.75% w/w, from about 0.02% to about 0.50% w/w, from about 0.02% to about 0.25% w/w, from about 0.02% to about 0.20% w/w, from about 0.02% to about 0.15% w/w, or from about 0.02% to about 0.10% w/w.

In some embodiments, the total amount of preservative is from about 0.50% to about 2.50% w/w, from about 0.50% to about 2.00% w/w, from about 0.50% to about 1.75% w/w, from about 0.50% to about 1.50% w/w, from about 0.50% to about 1.25% w/w, from about 0.50% to about 1.00% w/w, or from about 0.50% to about 0.75% w/w.

In some embodiments, the total amount of preservative is from about 0.10% to about 5.0% w/w. In some embodiments, the total amount of preservative is from about 0.10% to about 4.0% w/w. In some embodiments, the total amount of preservative is from about 0.10% to about 3.0% w/w. In some embodiments, the total amount of preservative is from about 0.10% to about 2.0% w/w. In some embodiments, the total amount of preservative is from about 0.10% to about 1.75% w/w. In some embodiments, the total amount of preservative is from about 0.10% to about 1.50% w/w. In some embodiments, the total amount of preservative is from about 0.10% to about 1.0% w/w. In some embodiments, the total amount of preservative is from about 0.10% to about 0.75% w/w. In some embodiments, the total amount of preservative is from about 0.10% to about 0.50% w/w. In some embodiments, the total amount of preservative is from about 0.10% to about 0.25% w/w. In some embodiments, the total amount of preservative is from about 0.10% to about 0.20% w/w. In some embodiments, the total amount of preservative is from about 0.10% to about 0.15% w/w.

In some embodiments, the vehicle in the second container further comprises at least one excipient selected from a preservative, a pH adjuster, a sweetener, a flavoring agent, a binder (i.e., a crystal growth inhibitor), a buffering agent, a co-solvent, a surfactant, an antioxidant, a polymer, an anti-caking agent, an antifoaming agent, a diluent, a lubricant, and a viscosity agent. In some embodiments, the vehicle in the second container further comprises at least one excipient selected from a pH adjuster, a sweetener, a flavoring agent, a binder, a buffering agent, an antifoaming agent, a diluent, and a lubricant. In some embodiments, the vehicle in the second container further comprises at least one sweetener. In some embodiments, the vehicle in the second container further comprises at least one flavoring agent. In some embodiments, the vehicle in the second container further comprises at least one binder. In some embodiments, the vehicle in the second container further comprises at least one pH adjuster.

In some embodiments, the total amount of excipient or excipients in the second container, other than the vehicle, is from about 0.005% to about 2.00% w/w.

In some embodiments, the total amount of excipient or excipients, other than the vehicle, is from about 0.01% to about 2.00% w/w, from about 0.01% to about 1.75% w/w, from about 0.01% to about 1.50% w/w, from about 0.01% to about 1.25% w/w, from about 0.01% to about 1.00% w/w, or from about 0.01% to about 0.75% w/w. In some embodiments, the total amount of excipient or excipients, other than the vehicle, is from about 0.01% to about 0.50% w/w. In some embodiments, the total amount of excipient or excipients, other than the vehicle, is from about 0.01% to about 0.25% w/w.

In some embodiments, the total amount of excipient or excipients, other than the vehicle, is from about 0.05% to about 2.00% w/w, from about 0.05% to about 1.75% w/w, from about 0.05% to about 1.50% w/w, from about 0.05% to about 1.25% w/w, from about 0.05% to about 1.00% w/w, or from about 0.05% to about 0.75% w/w. In some embodiments, the total amount of excipient or excipients, other than the vehicle, is from about 0.05% to about 0.50% w/w. In some embodiments, the total amount of excipient or excipients, other than the vehicle, is from about 0.05% to about 0.25% w/w.

In some embodiments, the total amount of excipient or excipients, other than a vehicle, is from about 0.10% to about 2.00% w/w, from about 0.10% to about 1.75% w/w, from about 0.10% to about 1.50% w/w, from about 0.10% to about 1.25% w/w, from about 0.10% to about 1.00% w/w, or from about 0.10% to about 0.75% w/w. In some embodiments, the total amount of excipient or excipients, other than the vehicle, is from about 0.10% to about 0.50% w/w. In some embodiments, the total amount of excipient or excipients, other than the vehicle, is from about 0.10% to about 0.25% w/w.

In some embodiments, after the contents of the first container is combined with the contents in the second container, the resultant liquid composition comprises an amount of fingolimod or salt thereof that is equivalent to from about 0.01 mg/mL to about 0.50 mg/mL of fingolimod. In some embodiments, the resultant liquid composition comprises an amount of fingolimod or salt thereof that is equivalent to from about 0.02 mg/mL to about 0.50 mg/mL, from about 0.03 mg/mL to about 0.50 mg/mL, from about 0.04 mg/mL to about 0.50 mg/mL, from about 0.05 mg/mL to about 0.50 mg/mL, from about 0.06 mg/mL to about 0.50 mg/mL, from about 0.07 mg/mL to about 0.50 mg/mL, from about 0.08 mg/mL to about 0.50 mg/mL, or from about 0.09 mg/mL to about 0.50 mg/mL of fingolimod. In some embodiments, the resultant liquid composition comprises an amount of fingolimod or salt thereof that is equivalent to from about 0.10 mg/mL to about 0.50 mg/mL of fingolimod.

In some embodiments, the resultant liquid composition comprises an amount of fingolimod or a salt thereof that is equivalent to from about 0.11 mg/mL to about 0.50 mg/mL, from about 0.12 mg/mL to about 0.50 mg/mL, from about 0.13 mg/mL to about 0.50 mg/mL, from about 0.14 mg/mL to about 0.50 mg/mL, from about 0.15 mg/mL to about 0.50 mg/mL, from about 0.16 mg/mL to about 0.50 mg/mL, from about 0.17 mg/mL to about 0.50 mg/mL, from about 0.18 mg/mL to about 0.50 mg/mL, or from about 0.19 mg/mL to about 0.50 mg/mL of fingolimod.

In some embodiments, the resultant liquid composition comprises an amount of fingolimod or a salt thereof that is equivalent to from about 0.20 mg/mL to about 0.50 mg/mL of fingolimod. In some embodiments, the resultant liquid composition comprises an amount of fingolimod or a salt thereof that is equivalent to from about 0.30 mg/mL to about 0.50 mg/mL of fingolimod. In some embodiments, the resultant liquid composition comprises an amount of fingolimod or a salt thereof that is equivalent to from about 0.40 mg/mL to about 0.50 mg/mL of fingolimod.

In some embodiments, the resultant liquid composition comprises an amount of fingolimod or salt thereof that is equivalent to from about 0.01 mg/mL to about 0.40 mg/mL, from about 0.02 mg/mL to about 0.40 mg/mL, from about 0.03 mg/mL to about 0.40 mg/mL, from about 0.04 mg/mL to about 0.40 mg/mL, from about 0.05 mg/mL to about 0.40 mg/mL, from about 0.06 mg/mL to about 0.40 mg/mL, from about 0.07 mg/mL to about 0.40 mg/mL, from about 0.08 mg/mL to about 0.40 mg/mL, or from about 0.09 mg/mL to about 0.40 mg/mL of fingolimod. In some embodiments, the resultant liquid composition comprises an amount of fingolimod or a salt thereof that is equivalent to from about 0.10 mg/mL to about 0.40 mg/mL of fingolimod.

In some embodiments, the resultant liquid composition comprises an amount of fingolimod or a salt thereof that is equivalent to from about 0.11 mg/mL to about 0.40 mg/mL, from about 0.12 mg/mL to about 0.40 mg/mL, from about 0.13 mg/mL to about 0.40 mg/mL, from about 0.14 mg/mL to about 0.40 mg/mL, from about 0.15 mg/mL to about 0.40 mg/mL, from about 0.16 mg/mL to about 0.40 mg/mL, from about 0.17 mg/mL to about 0.40 mg/mL, from about 0.18 mg/mL to about 0.40 mg/mL, or from about 0.19 mg/mL to about 0.40 mg/mL of fingolimod.

In some embodiments, the resultant liquid composition comprises an amount of fingolimod or a salt thereof that is equivalent to from about 0.20 mg/mL to about 0.40 mg/mL of fingolimod. In some embodiments, the resultant liquid composition comprises an amount of fingolimod or a salt thereof that is equivalent to from about 0.30 mg/mL to about 0.40 mg/mL of fingolimod.

In some embodiments, the resultant liquid composition comprises an amount of fingolimod or salt thereof that is equivalent to from about 0.01 mg/mL to about 0.30 mg/mL, from about 0.02 mg/mL to about 0.30 mg/mL, from about 0.03 mg/mL to about 0.30 mg/mL, from about 0.04 mg/mL to about 0.30 mg/mL, from about 0.05 mg/mL to about 0.30 mg/mL, from about 0.06 mg/mL to about 0.30 mg/mL, from about 0.07 mg/mL to about 0.30 mg/mL, from about 0.08 mg/mL to about 0.30 mg/mL, or from about 0.09 mg/mL to about 0.30 mg/mL of fingolimod. In some embodiments, the resultant liquid composition comprises an amount of fingolimod or a salt thereof that is equivalent to from about 0.10 mg/mL to about 0.30 mg/mL of fingolimod.

In some embodiments, the resultant liquid composition comprises an amount of fingolimod or a salt thereof that is equivalent to from about 0.11 mg/mL to about 0.30 mg/mL, from about 0.12 mg/mL to about 0.30 mg/mL, from about 0.13 mg/mL to about 0.30 mg/mL, from about 0.14 mg/mL to about 0.30 mg/mL, from about 0.15 mg/mL to about 0.30 mg/mL, from about 0.16 mg/mL to about 0.30 mg/mL, from about 0.17 mg/mL to about 0.30 mg/mL, from about 0.18 mg/mL to about 0.30 mg/mL, from about 0.19 mg/mL to about 0.30 mg/mL, or from about 0.20 mg/mL to about 0.30 mg/mL of fingolimod.

In some embodiments, the resultant liquid composition comprises an amount of fingolimod or salt thereof that is equivalent to from about 0.01 mg/mL to about 0.20 mg/mL, from about 0.02 mg/mL to about 0.20 mg/mL, from about 0.03 mg/mL to about 0.20 mg/mL, from about 0.04 mg/mL to about 0.20 mg/mL, from about 0.05 mg/mL to about 0.20 mg/mL, from about 0.06 mg/mL to about 0.20 mg/mL, from about 0.07 mg/mL to about 0.20 mg/mL, from about 0.08 mg/mL to about 0.20 mg/mL, or from about 0.09 mg/mL to about 0.20 mg/mL of fingolimod. In some embodiments, the composition comprises an amount of fingolimod or a salt thereof that is equivalent to from about 0.10 mg/mL to about 0.20 mg/mL of fingolimod.

In some embodiments, the resultant liquid composition comprises an amount of fingolimod or a salt thereof that is equivalent to from about 0.11 mg/mL to about 0.20 mg/mL, from about 0.12 mg/mL to about 0.20 mg/mL, from about 0.13 mg/mL to about 0.20 mg/mL, from about 0.14 mg/mL to about 0.20 mg/mL, from about 0.15 mg/mL to about 0.20 mg/mL, from about 0.16 mg/mL to about 0.20 mg/mL, from about 0.17 mg/mL to about 0.20 mg/mL, from about 0.18 mg/mL to about 0.20 mg/mL, or from about 0.19 mg/mL to about 0.20 mg/mL of fingolimod.

In some embodiments, the resultant composition comprises an amount of fingolimod or a salt thereof that is equivalent to about 0.01 mg/mL, about 0.02 mg/mL, about 0.03 mg/mL, about 0.04 mg/mL, about 0.05 mg/mL, about 0.06 mg/mL, about 0.07 mg/mL, about 0.08 mg/mL, or about 0.09 mg/mL of fingolimod. In some embodiments, the composition comprises an amount of fingolimod or a salt thereof that is equivalent to about 0.10 mg/mL of fingolimod.

In some embodiments, the resultant liquid composition comprises an amount of fingolimod or a salt thereof that is equivalent to about 0.11 mg/mL, about 0.12 mg/mL, about 0.13 mg/mL, about 0.14 mg/mL, about 0.15 mg/mL, about 0.16 mg/mL, about 0.17 mg/mL, about 0.18 mg/mL, about 0.19 mg/mL, or about 0.20 mg/mL of fingolimod.

In some embodiments, the resultant liquid composition comprises an amount of fingolimod or a salt thereof that is equivalent to about 0.21 mg/mL, about 0.22 mg/mL, about 0.23 mg/mL, about 0.24 mg/mL, about 0.25 mg/mL, about 0.26 mg/mL, about 0.27 mg/mL, about 0.28 mg/mL, about 0.29 mg/mL, or about 0.30 mg/mL of fingolimod.

In some embodiments, the resultant liquid composition comprises an amount of fingolimod or a salt thereof that is equivalent to about 0.31 mg/mL, about 0.32 mg/mL, about 0.33 mg/mL, about 0.34 mg/mL, about 0.35 mg/mL, about 0.36 mg/mL, about 0.37 mg/mL, about 0.38 mg/mL, about 0.39 mg/mL, or about 0.40 mg/mL of fingolimod.

In some embodiments, the resultant liquid composition comprises an amount of fingolimod or a salt thereof that is equivalent to about 0.41 mg/mL, about 0.42 mg/mL, about 0.43 mg/mL, about 0.44 mg/mL, about 0.45 mg/mL, about 0.46 mg/mL, about 0.47 mg/mL, about 0.48 mg/mL, about 0.49 mg/mL, or about 0.50 mg/mL of fingolimod.

In some embodiments, the resultant liquid composition is stable, as described herein. In some embodiments, the resultant liquid composition is shelf stable.

In some embodiments, the kit further comprises a tool for scraping the fingolimod or a salt thereof in the first container into the vehicle in the second container, or for scraping the vehicle in the second container into the fingolimod or a salt thereof in the first container.

In some embodiments, the kit further comprises a measuring tool (e.g., a cup or syringe) that may be used for measuring and/or administration of the composition. In some embodiments, the use of the tool allows a precise dosage to be obtained.

In some embodiments, disclosed herein is a packaged product comprising a liquid composition as described herein in a container. In some embodiments, the packaged product may be in a second container. In some embodiments, the container(s) reduce exposure of the composition to light.

In some embodiments, the container is a bottle. In some embodiments, the container is a vial. In some embodiments, the container is a pouch. In some embodiments, the container is a bag. In some embodiments, the container is a box. In some embodiments, the container is a monocarton.

In some embodiments, the container is a clear container. In some embodiments, the container is an opaque container. In some embodiments, the container is a colored container. In some embodiments, the container is a white-colored container. In some embodiments, the container is an amber-colored container. In some embodiments, the container is an orange-colored container.

In some embodiments, the container has a fill volume of from about 10 mL to about 500 mL, about 10 mL to about 450 mL, about 10 mL to about 400 mL, about 10 mL to about 350 mL, about 10 mL to about 300 mL, or about 10 mL to about 250 mL. In some embodiments, the container has a fill volume of from about 10 mL to about 200 mL, from about 10 mL to about 150 mL, from about 10 mL to about 100 mL, or from about 10 mL to about 50 mL. In some embodiments, the container has a fill volume of from about 50 mL to about 250 mL, from about 50 mL to about 200 mL, from about 50 mL to about 150 mL, or from about 50 mL to about 100 mL. In some embodiments, the container has a fill volume of from about 100 mL to about 250 mL, or from about 100 mL to about 200 mL. In some embodiments, the container has a fill volume of from about 100 mL to about 150 mL. In some embodiments, the container has a fill volume of from about 150 mL to about 250 mL. In some embodiments, the container has a fill volume of from about 150 mL to about 200 mL. In some embodiments, the container has a fill volume of from about 200 mL to about 250 mL.

In some embodiments, the container has a fill volume of about 50 mL, about 100 mL, about 150 mL, about 200 mL, about 250 mL, about 300 mL, about 350 mL, about 400 mL, about 450 mL, or about 500 mL. In some embodiments, the container has a fill volume of about 100 mL. In some embodiments, the container has a fill volume of about 150 mL. In some embodiments, the container has a fill volume of about 200 mL. Therapeutic Methods and Uses

In some embodiments, the present disclosure relates to a method of treating a subject with multiple sclerosis (MS) comprising administering to the subject in need thereof the liquid composition as described herein.

In some embodiments, the present disclosure relates to a method of treating a subject with multiple sclerosis (MS), the method comprising combining the contents of the first container and the contents of the second container in the kit as described herein and administering the resultant combination to the subject.

In some embodiments, the multiple sclerosis (MS) is a relapsing form. In some embodiments, the relapsing form is selected from clinically isolated syndrome, relapsing-remitting disease, and active secondary progressive disease. In some embodiments, the relapsing form is clinically isolated syndrome. In some embodiments, the relapsing form is relapsing-remitting disease. In some embodiments, the relapsing form is active secondary progressive disease.

In some embodiments, the treatment is prophylactic treatment. In some embodiments, the treatment is chronic treatment.

In some embodiments, the subject weighs more than 40 kg. In some embodiments, the subject weighs less than or equal to 40 kg.

In some embodiments, the subject is a pediatric or geriatric patient. In some embodiments, the subject is a geriatric patient. In some embodiments, the subject is a pediatric patient. In some embodiments, the pediatric patient is 10 years of age and older. In some embodiments, a pediatric patient is about 10 years old to about 18 years old. In some embodiments, a pediatric patient is about 10 years old to about 12 years old. In some embodiments, a pediatric patient is about 10 years old to about 16 years old. In some embodiments, a pediatric patient is about 10 years old. In some embodiments, a pediatric patient is about 11 years old. In some embodiments, a pediatric patient is about 12 years old. In some embodiments, a pediatric patient is about 13 years old. In some embodiments, a pediatric patient is about 14 years old. In some embodiments, a pediatric patient is about 15 years old. In some embodiments, a pediatric patient is about 16 years old. In some embodiments, a pediatric patient is about 17 years old. In some embodiments, a pediatric patient is about 18 years old.

In some embodiments, in the methods described herein, the composition is administered in a dose of from about 0.10 mg/mL to about 1.00 mg/mL of fingolimod. In some embodiments, in the methods described herein, the composition is administered in a dose of from about 0.10 mg/mL to about 0.75 mg/mL, from about 0.10 mg/mL to about 0.70 mg/mL, from about 0.10 mg/mL to about 0.65 mg/mL, from about 0.10 mg/mL to about 0.60 mg/mL, from about 0.10 mg/mL to about 0.55 mg/mL, from about 0.10 mg/mL to about 0.50 mg/mL, from about 0.10 mg/mL to about 0.45 mg/mL, from about 0.10 mg/mL to about 0.40 mg/mL, from about 0.10 mg/mL to about 0.35 mg/mL, from about 0.10 mg/mL to about 0.30 mg/mL, from about 0.10 mg/mL to about 0.25 mg/mL, from about 0.10 mg/mL to about 0.20 mg/mL of fingolimod.

In some embodiments, the composition is administered in a dose of from about 0.25 mg/mL to about 1.00 mg/mL of fingolimod. In some embodiments, in the methods described herein, the composition is administered in a dose of from about 0.25 mg/mL to about 0.75 mg/mL, from about 0.25 mg/mL to about 0.70 mg/mL, from about 0.25 mg/mL to about 0.65 mg/mL, from about 0.25 mg/mL to about 0.60 mg/mL, from about 0.25 mg/mL to about 0.55 mg/mL, from about 0.25 mg/mL to about 0.50 mg/mL, from about 0.25 mg/mL to about 0.45 mg/mL, from about 0.25 mg/mL to about 0.40 mg/mL, from about 0.25 mg/mL to about 0.35 mg/mL, or from about 0.25 mg/mL to about 0.30 mg/mL of fingolimod.

In some embodiments, the composition is administered in a dose of from about 0.50 mg/mL to about 1.00 mg/mL of fingolimod. In some embodiments, in the methods described herein, the composition is administered in a dose of from about 0.50 mg/mL to about 0.75. mg/mL of fingolimod.

In some embodiments, in the methods described herein, the composition is administered in a dose of about 0.10 mg/mL of fingolimod. In some embodiments, in the methods described herein, the composition is administered in a dose of about 0.15 mg/mL of fingolimod. In some embodiments, in the methods described herein, the composition is administered in a dose of about 0.20 mg/mL of fingolimod.

In some embodiments, in the methods described herein, the composition is administered in a dose of about 0.25 mg/mL of fingolimod. In some embodiments, in the methods described herein, the composition is administered in a dose of about 0.30 mg/mL of fingolimod. In some embodiments, in the methods described herein, the composition is administered in a dose of about 0.35 mg/mL of fingolimod. In some embodiments, in the methods described herein, the composition is administered in a dose of about 0.40 mg/mL of fingolimod. In some embodiments, in the methods described herein, the composition is administered in a dose of about 0.45 mg/mL of fingolimod.

In some embodiments, in the methods described herein, the composition is administered in a dose of about 0.50 mg/mL of fingolimod. In some embodiments, in the methods described herein, the composition is administered in a dose of about 0.55 mg/mL of fingolimod. In some embodiments, in the methods described herein, the composition is administered in a dose of about 0.60 mg/mL of fingolimod. In some embodiments, in the methods described herein, the composition is administered in a dose of about 0.65 mg/mL of fingolimod. In some embodiments, in the methods described herein, the composition is administered in a dose of about 0.70 mg/mL of fingolimod.

In some embodiments, in the methods described herein, the composition is administered in a dose of about 0.75 mg/mL of fingolimod. In some embodiments, in the methods described herein, the composition is administered in a dose of about 0.80 mg/mL of fingolimod. In some embodiments, in the methods described herein, the composition is administered in a dose of about 0.85 mg/mL of fingolimod. In some embodiments, in the methods described herein, the composition is administered in a dose of about 0.90 mg/mL of fingolimod. In some embodiments, in the methods described herein, the composition is administered in a dose of about 0.95 mg/mL of fingolimod. In some embodiments, in the methods described herein, the composition is administered in a dose of about 1.00 mg/mL of fingolimod.

In some embodiments, the composition is administered orally. In some embodiments, the composition is administered as a single dose once per day. In some embodiments, the composition is administered orally as a single dose once per day.

In some embodiments, the composition is administered with or without food. In some embodiments, the composition is administered with food. In some embodiments, the composition is administered without food.

In some embodiments, the subject has difficulty and/or pain with swallowing. In some embodiments, the subject suffers from dysphagia. In some embodiments, the subject prefers a liquid oral medication over a solid oral medication.

In some embodiments, the subject does not suffer from chickenpox, also known as varicella-zoster virus (VZV), at the time of treatment with fingolimod. In some embodiments, the subject has no confirmed medical history of chickenpox. In some embodiments, the subject has been vaccinated against chickenpox. In some embodiments, the subject has been vaccinated against VZV at least about 1 month prior to treatment with fingolimod.

In some embodiments, the present disclosure relates to a method of treating a subject with a relapsing form of multiple sclerosis (MS), the method comprising administering a liquid composition as described herein in a dose of about 0.5 mg of fingolimod once daily, wherein the subject is 10 years of age and older and weighs more than 40 kg. In some embodiments, the relapsing form of multiple sclerosis (MS) is selected from clinically isolated syndrome, relapsing-remitting disease, and active secondary progressive disease.

In some embodiments, the present disclosure relates to a method of treating a subject with a relapsing form of multiple sclerosis (MS), the method comprising administering a liquid composition as described herein in a dose of about 0.25 mg of fingolimod once daily, wherein the subject is 10 years of age and older and weighs less than or equal to 40 kg. In some embodiments, the relapsing form of multiple sclerosis (MS) is selected from clinically isolated syndrome, relapsing-remitting disease, and active secondary progressive disease.

In some embodiments, the method comprises a first-dose monitoring process comprising:
(a) observing the subject for bradycardia for at least 6 hours, monitoring pulse and blood pressure hourly, conducting electrocardiograms (ECGs) both prior to dosing and at the end of an observation period;
(b) monitoring until resolution if the subject's heart rate is greater than 45 beats per minute (bpm): when the subject is an adult, greater than 55 bpm; when the subject is aged 12 years or less, or greater than 60 bpm if the subject is aged 10 to below 12 years, atrioventricular (AV) block, or if lowest postdose heart rate is at the end of the observation period;
(c) monitoring symptomatic bradycardia with ECG until resolved, continuing overnight if intervention is required, repeating first-dose monitoring for second dose; and/or
(d) observing the subject overnight if at high risk of symptomatic bradycardia, heart block, prolonged QTc interval, or if taking drugs with known risk of Torsades de Pointes (TdP).

### Methods of Preparation

In some embodiments, the present disclosure relates to a method of preparing a liquid composition as described herein.

In some embodiments, the method of preparing a liquid composition as described herein comprises combining fingolimod or a salt thereof with a vehicle. In some embodiments, the vehicle comprises at least one additional excipient. In some embodiments, the method comprises a first step of combining a vehicle with at least one excipient. In some embodiments, the method comprises a second step of combining the vehicle with the at least one excipient with fingolimod or a salt thereof.

In some embodiments, the method of preparing a liquid composition as described herein comprises: (a) combining at least one excipient (for example, selected from sweeteners and preservatives) in a vehicle (for example, water) to generate a first solution; (b) adding fingolimod or a salt thereof to the first solution to generate a second solution; and (c) adjusting the pH of the second solution (for example, to from about 3 to about 6). In some embodiments, the second solution is diluted and the final pH of the second solution is from about 3 to about 6.

In some embodiments, an inert atmosphere is maintained during the preparation of a liquid composition as described herein.

In some embodiments, the method of preparing a liquid composition as described herein comprises combining the contents of the first container in a kit as described herein with the contents of the second container in a kit as described herein.

In some embodiments, disclosed herein is a method of preparing a packaged product as described herein, comprising placing the liquid composition into the container (such as a bottle). In some embodiments, the method further comprises sealing the container. In some embodiments, the method comprises placing the packaged product into a second container (such as a box).

All documents cited are, in relevant part, incorporated herein by reference. The citation of any document is not to be construed as an admission that it is prior art.

### ENUMERATED EMBODIMENTS

Embodiment 1. An oral liquid pharmaceutical composition comprising; (a) fingolimod or a pharmaceutically acceptable salt thereof; (b) at least one pharmaceutically acceptable vehicle; and (c) at least one pharmaceutically acceptable excipient.
Embodiment 2. An oral liquid pharmaceutical composition comprising fingolimod or a pharmaceutically acceptable salt thereof, wherein the composition is a stable aqueous solution.
Embodiment 3. An oral liquid pharmaceutical composition comprising fingolimod or a pharmaceutically acceptable salt thereof, wherein the composition comprises at least one pH adjuster selected from HCl and NaOH.
Embodiment 4. The oral liquid pharmaceutical composition according to any of the preceding embodiments, wherein the composition comprises at least one preservative selected from a paraben, sodium propionate, and benzyl alcohol.
Embodiment 5. The oral liquid pharmaceutical composition according to any of the preceding embodiments, wherein the concentration of fingolimod is about 0.1 mg/mL.
Embodiment 6. The oral liquid pharmaceutical composition according to any of the preceding embodiments, wherein the composition comprises a pharmaceutically acceptable vehicle selected from a group consisting of water, alcohols, glycols, dimethylacetamide N-methylpyrollidone, dimethyl sulfoxide, ringer's solution, isotonic sodium chloride solution, glycerine, alcohols, and polyethylene glycol, or their mixtures thereof.
Embodiment 7. The oral liquid pharmaceutical composition according to any of the preceding embodiments, wherein the composition comprises a pharmaceutically acceptable vehicle is not propylene glycol.
Embodiment 8. The oral liquid pharmaceutical composition according to any of the preceding embodiments, wherein the composition is stable at room temperature.
Embodiment 9. The oral liquid pharmaceutical composition according to any of the preceding embodiments, wherein the composition is substantially free of complexing agents.
Embodiment 10. The oral liquid pharmaceutical composition according to any of the preceding embodiments, wherein the composition is substantially free of cyclodextrin or derivatives thereof.
Embodiment 11. The oral liquid pharmaceutical composition according to any of the preceding embodiments, wherein the composition is ready to use composition and does not need further dilution.
Embodiment 12. An oral pharmaceutical composition consisting of: (a) fingolimod or a pharmaceutically acceptable salt thereof at a concentration of from about 0.05 mg/mL to about 10 mg/mL; (b) a pharmaceutically acceptable vehicle; and (c) optionally at least one pharmaceutically acceptable excipient.
Embodiment 13. An oral pharmaceutical composition consisting of: (a) fingolimod or a pharmaceutically acceptable salt thereof at a concentration of from about 0.05 mg/mL to about 10 mg/mL; (b) a pharmaceutically acceptable vehicle; (c) at least one preservative; (d) optionally at least one sweetening agent; (e) at least one pH adjusting agent; (f) at least one buffering agent and (g) optionally at least one other pharmaceutically acceptable excipient.
Embodiment 14. An oral pharmaceutical composition essentially consisting of: (a) fingolimod or a pharmaceutically acceptable salt thereof at a concentration of from about 0.05 mg/mL to about 10 mg/mL; (b) a pharmaceutically acceptable vehicle; (c) at least one preservative; (d) optionally at least one sweetening agent; (e) at least one pH adjusting agents; and (f) at least one buffering agent.
Embodiment 15. An oral liquid pharmaceutical composition comprising;
   (a) about 0.0112% to about 0.1% w/w of fingolimod or a pharmaceutically acceptable salt thereof;
   (b) about 0.01% to about 3% w/w of at least one preservative;
   (c) optionally about 0.01% to about 3% w/w of at least one sweetening agent;
   (d) about 0.1% to about 5% w/w of at least one pH adjusting agent;
   (e) about 90% to about 99.9% w/w of a pharmaceutically acceptable vehicle; and
   (f) optionally at least one pharmaceutically acceptable excipient selected from buffering agents, complexing agents, binders, and antioxidants;
   wherein the pH of the composition is in range of about 3 to about 6.
Embodiment 16. An oral liquid pharmaceutical solution comprising;
   (a) about 0.0112% to about 0.1% w/w of fingolimod or a pharmaceutically acceptable salt thereof;
   (b) about 0.01% to about 3% w/w of at least one preservative;
   (c) optionally about 0.01% to about 3% w/w of at least one sweetening agent;
   (d) about 0.1% to about 5% w/w of at least one pH adjusting agent;
   (e) about 90% to about 99.9% w/w of a pharmaceutically acceptable vehicle; and
   (f) optionally at least one pharmaceutically acceptable excipient selected from buffering agents, complexing agents, binders, and antioxidants;
   wherein the pH of the solution is in range of about 3 to about 6.
Embodiment 17. The oral liquid pharmaceutical composition of any one of the preceding embodiments, wherein the composition is stable after at least one freeze-thaw cycle.
Embodiment 18. The oral liquid pharmaceutical composition of any one of the preceding embodiments, wherein the composition is photostable.
Embodiment 19. An oral liquid pharmaceutical composition comprising fingolimod or a salt thereof, wherein the composition is stable after at least one freeze-thaw cycle, for example after at least three freeze-thaw cycles, for example after at least five freeze-thaw cycles.
Embodiment 20. The oral liquid pharmaceutical composition of any one of the preceding embodiments, wherein each freeze-thaw cycle comprises about 48 hours at about −20° C., followed by about 48 hours at room temperature.
Embodiment 21. The oral liquid pharmaceutical composition of any one of the preceding embodiments, wherein the amount of fingolimod in the composition after each freeze-thaw cycle is from about 80% to about 100% w/w.
Embodiment 22. The oral liquid pharmaceutical composition of any one of the preceding embodiments, wherein the composition is stable for at least 12 months at about 25° C. and about 60% RH.
Embodiment 23. The oral liquid pharmaceutical composition of any one of the preceding embodiments, wherein the composition is stable for at least 6 months at about 40° C. and about 75% RH.
Embodiment 24. The oral liquid pharmaceutical composition of any one of the preceding embodiments, wherein the composition is photostable.
Embodiment 25. An oral liquid pharmaceutical composition comprising fingolimod or a pharmaceutically acceptable salt thereof, wherein the composition is photostable.
Embodiment 26. The oral liquid pharmaceutical composition of any one of the preceding embodiments, wherein the composition is photostable when exposed to an overall illumination of about 1.2 million lux hours.
Embodiment 27. The oral liquid pharmaceutical composition of any one of the preceding embodiments, wherein the composition is photostable when exposed to an integrated near ultraviolet energy of about 200-watt hours/square meter.
Embodiment 28. The oral liquid pharmaceutical composition of embodiment 27 or 28, wherein the amount of fingolimod in the composition after exposure to illumination or an integrated near ultraviolet energy is from about 80% to about 100% w/w.
Embodiment 29. The oral liquid pharmaceutical composition of any one of the preceding embodiments, wherein the composition comprises an amount of fingolimod or salt thereof that is equivalent to from about 0.01 mg/mL to about 0.50 mg/mL of fingolimod.
Embodiment 30. The oral liquid pharmaceutical composition of the immediately preceding embodiment, wherein the composition comprises an amount of fingolimod or a salt thereof that is equivalent to from about 0.10 mg/mL to about 0.20 mg/mL of fingolimod.
Embodiment 31. The oral liquid pharmaceutical composition of the immediately preceding embodiment, wherein the composition comprises an amount of fingolimod or a salt thereof that is equivalent to about 0.10 mg/mL of fingolimod.
Embodiment 32. The oral liquid pharmaceutical composition of any one of the preceding embodiments, wherein the composition comprises fingolimod and/or fingolimod hydrochloride.
Embodiment 33. The oral liquid pharmaceutical composition of any one of the preceding embodiments, wherein the composition comprises at least two preservatives independently selected from a paraben, sodium propionate, and benzyl alcohol.
Embodiment 34. The oral liquid pharmaceutical composition of any one of the preceding embodiments, wherein the total amount of preservative in the composition is from about 0.01% to about 2.50% w/w.
Embodiment 35. The oral liquid pharmaceutical composition of the immediately preceding embodiment, wherein the total amount of preservative in the composition is from about 0.02% to about 0.25% w/w.
Embodiment 36. The oral liquid pharmaceutical composition of any one of the preceding embodiments, wherein the composition comprises at least two parabens.
Embodiment 37. The oral liquid pharmaceutical composition of the immediately preceding embodiment, wherein each paraben is independently selected from methyl paraben and propyl paraben.
Embodiment 38. The oral liquid pharmaceutical composition of any one of the preceding embodiments, wherein the composition comprises at least one excipient selected from a sweetener, a flavoring agent, a binder (i.e., crystal growth inhibitor), and an alkalizer.
Embodiment 39. The oral liquid pharmaceutical composition of the immediately preceding embodiment, wherein the sweetener is sucralose or xylitol.
Embodiment 40. The oral liquid pharmaceutical composition of embodiment 38 or 39, wherein the total amount of sweetener in the composition is from about 0.005% to about 0.05% w/w.
Embodiment 41. The oral liquid pharmaceutical composition of the immediately preceding embodiment, wherein the total amount of sweetener in the composition is from about 0.01% to about 0.025% w/w.
Embodiment 42. The oral liquid pharmaceutical composition of embodiment 38, wherein the binder is hypromellose or povidone.
Embodiment 43. The oral liquid pharmaceutical composition of embodiment 38 or 42, wherein the total amount of the binder in the composition is from about 0.5% to about 1.5% w/w.
Embodiment 44. The oral liquid pharmaceutical composition of the immediately preceding embodiment, wherein the total amount of the binder in the composition is about 1.0% w/w.
Embodiment 45. The oral liquid pharmaceutical composition of embodiment 38, wherein the alkalizer is meglumine or L-Arginine.
Embodiment 46. The oral liquid pharmaceutical composition of embodiment 38 or 45, wherein the total amount of alkalizer in the composition is from about 0.1% to about 0.5% w/w.
Embodiment 47. The oral liquid pharmaceutical composition of the immediately preceding embodiment, wherein the total amount of alkalizer in the composition is about 0.25% w/w.
Embodiment 48. The oral liquid pharmaceutical composition of any one of the preceding embodiments, wherein the composition has a pH of about 2.0 to about 9.0, for example from about 3.5 to about 6.0.
Embodiment 49. The oral liquid pharmaceutical composition of the immediately preceding embodiment, wherein the composition has a pH of about 4.0 to about 7.0.
Embodiment 50. The oral liquid pharmaceutical composition of the immediately preceding embodiment, wherein the composition has a pH of about 4.5 to about 6.5.
Embodiment 51. The oral liquid pharmaceutical composition of the immediately preceding embodiment, wherein the composition has a pH of about 4.5.
Embodiment 52. The oral liquid pharmaceutical composition of any one of the preceding embodiments, wherein the composition is a solution.
Embodiment 53. The oral liquid pharmaceutical composition of any one of the preceding embodiments, wherein the composition is ready to use.
Embodiment 54. The oral liquid pharmaceutical composition of any one of the preceding embodiments, wherein the composition is in the form of an oral dosage form.
Embodiment 55. The oral liquid pharmaceutical composition of any one of the preceding embodiments, wherein the composition is stable after being stored at about 25° C. and about 60% relative humidity for about one, about three, about six, or about 12 months.
Embodiment 56. The oral liquid pharmaceutical composition of any one of the preceding embodiments, wherein the composition is stable after being stored at about 40° C. and about 75% relative humidity for about one, about three, about six, or about 12 months.
Embodiment 57. A kit comprising:
(i) a first container comprising fingolimod or a salt thereof, or the oral liquid pharmaceutical composition according to any one of embodiments 1-56 or 74-95; and
(ii) a second container comprising a pharmaceutically acceptable vehicle.
Embodiment 58. The kit according to the immediately preceding embodiment, wherein the first container comprises fingolimod or a salt thereof.
Embodiment 59. The kit according to the immediately preceding embodiment, wherein the first container further comprises at least one excipient.
Embodiment 60. The kit according to any one of embodiments 57-59, wherein the fingolimod or a salt thereof is in the form of a powder.
Embodiment 61. The kit according to any one of embodiments 57-60, wherein the first container comprises fingolimod hydrochloride.
Embodiment 62. The kit according to any one of embodiments 57-61, wherein the vehicle in the second container comprises water.
Embodiment 63. The kit according to the immediately preceding embodiment, wherein the vehicle in the second container further comprises a preservative.
Embodiment 64. The kit according to embodiment 62 or 63, wherein the vehicle in the second container further comprises at least one excipient selected from a sweetener, a flavoring agent, a binder, and a pH adjuster such as an alkalizer.
Embodiment 65. The kit according to any one of embodiments 57-64, wherein the second container comprises from about 50 mL to about 500 mL of the vehicle.
Embodiment 66. The kit according to the immediately preceding embodiment, wherein the second container comprises about 150 mL of the vehicle.
Embodiment 67. A method of treating a subject with multiple sclerosis (MS) comprising administering to the subject in need thereof the oral liquid pharmaceutical composition according to any one of embodiments 1-56 or 74-95.
Embodiment 68. A method of treating a subject with multiple sclerosis (MS), the method comprising combining the contents of the first container and the contents of the second container in the kit according to any one of embodiments 57-66 and administering the resultant combination to the subject.
Embodiment 69. The method of embodiment 67 or 68, wherein the multiple sclerosis (MS) is a relapsing form.
Embodiment 70. The method of the immediately preceding embodiment, wherein the relapsing form is selected from clinically isolated syndrome, relapsing-remitting disease, and/or active secondary progressive disease.
Embodiment 71. The method of any one of embodiments 67-70, wherein the subject is a pediatric or geriatric patient.
Embodiment 72. The method of the immediately preceding embodiment, wherein the pediatric patient is at least 10 years of age.
Embodiment 73. A method of preparing an oral liquid pharmaceutical composition according to any one of embodiments 1-56 or 74-95, the method comprising: (a) combining at least one excipient (for example, selected from sweeteners and preservatives) in a vehicle (for example, water) to generate a first solution; (b) adding fingolimod or a salt thereof to the first solution to generate a second solution; and (c) adjusting the pH of the second solution (for example, to from about 3 to about 6).
Embodiment 74. A stable oral liquid pharmaceutical composition comprising:
   (a) therapeutically effective amount of fingolimod or a pharmaceutically acceptable salt thereof;
   (b) a pharmaceutically acceptable liquid vehicle selected from group consisting of water, ringer's solution, isotonic sodium chloride solution, glycerin, propylene glycol, ethanol, polyethylene glycol or a combination thereof;
   (c) optionally one or more excipients selected from group consisting of pH adjusting agents, preservatives, sweetening agents, crystal growth inhibitors, buffering agents, complexing agents, thickening agents, stabilizers, solubilizers, anti-oxidants, flavoring agents, coloring agents or combination thereof;
   wherein pH of said composition ranges from about 3.0 to about 6.0; and
   wherein said composition is stable for at least 6 months when stored at about 25° C. and about 60% relative humidity (RH).
Embodiment 75. The oral liquid pharmaceutical composition according to embodiment 74, wherein the pharmaceutical composition is a solution, a suspension or an emulsion.
Embodiment 76. The oral liquid pharmaceutical composition according to embodiment 74 or 73, wherein the composition is an aqueous solution.
Embodiment 77. The oral liquid pharmaceutical composition according to any one of embodiments 74-76, wherein the pH adjusting agent is selected from HCl and NaOH or a combination thereof.
Embodiment 78. The oral liquid pharmaceutical composition according to any one of embodiments 74-77, wherein the preservative is in the range of about 0% to about 3% w/w.
Embodiment 79. The oral liquid pharmaceutical composition according to any one of embodiments 74-78, wherein the preservative is selected from methyl paraben, propyl paraben, sodium propionate, benzyl alcohol, potassium sorbate, sodium benzoate, butylated hydroxyanisole, butylated hydroxytoluene, chlorobutanol, phenyl ethyl alcohol or a combination thereof.
Embodiment 80. The oral liquid pharmaceutical composition according to any one of embodiments 74-79, wherein the sweetening agent is in the range of about 0% to about 3% w/w.
Embodiment 81. The oral liquid pharmaceutical composition according to any one of embodiments 74-80, wherein the sweetening agent is selected from sucralose, trehalose, xylose, dextrose, tagatose, glycerol, lactitol, saccharine or the corresponding sodium, potassium or calcium salt, cyclamate or the corresponding sodium or calcium salt, aspartame, or acesulfame or the potassium salt thereof or a combination thereof.
Embodiment 82. The oral liquid pharmaceutical composition according to any one of embodiments 74-81, wherein the crystal growth inhibitor is in the range of about 0% to about 5% w/w.
Embodiment 83. The oral liquid pharmaceutical composition according to any one of embodiments 74-82, wherein the crystal growth inhibitor is selected from hydroxypropyl methylcellulose, microcrystalline cellulose, hydroxypropyl methylcellulose acetate succinate, povidone or a combination thereof.
Embodiment 84. The oral liquid pharmaceutical composition according to any one of embodiments 74-83, wherein the alkalizer is selected from meglumine or L-arginine or a combination thereof.
Embodiment 85. The oral liquid pharmaceutical composition according to any one of embodiments 74-84, wherein the buffering agent is in the range of about 0% to about 5% w/w.
Embodiment 86. The oral liquid pharmaceutical composition according to any one of embodiments 74-85, wherein the buffering agent is selected from sodium citrate, sodium acetate, sodium phosphate, potassium phosphate, tris, sodium succinate, histidine, glycine, arginine, sodium malate, sodium tartrate, sodium benzoate, gluconic acid, ascorbic acid, triethanolamine or a combination thereof.
Embodiment 87. The oral liquid pharmaceutical composition according to any one of embodiments 74-86, wherein the composition is a ready-to-use composition and does not need further dilution prior to administration.
Embodiment 88. The oral liquid pharmaceutical composition according to any one of embodiments 74-87, wherein the composition comprises fingolimod or fingolimod hydrochloride at a concentration in the range of about 0.05 mg/mL to about 10 mg/mL.
Embodiment 89. The oral liquid pharmaceutical composition according to any one of embodiments 74-88, wherein the composition comprises fingolimod or fingolimod hydrochloride at a concentration of about 0.1 mg/mL.
Embodiment 90. The oral liquid pharmaceutical composition according to any one of embodiments 74-89, wherein the composition has a pH of about 3.0 to about 5.0.
Embodiment 91. The oral liquid pharmaceutical composition according to any one of embodiments 74-90, wherein the composition has a pH of about 4.5.
Embodiment 92. The oral liquid composition according to any one of embodiments 74-91, wherein the composition is stable after at least two freeze-thaw cycles.
Embodiment 93. The oral liquid pharmaceutical composition according to any one of embodiments 74-92, wherein the level of total impurities in the composition is less than about 1% w/w as measured by HPLC, when said solution is stored at about 40° C. and about 75% relative humidity (RH) for at least 6 months.
Embodiment 94. A stable oral liquid pharmaceutical composition comprising:
   (a) about 0.001% to about 0.5% w/w of fingolimod or a pharmaceutically acceptable salt thereof;
   (b) about 10% to 99.99% w/w of a pharmaceutically acceptable vehicle; and
   (c) optionally one or more excipients selected from group consisting of pH adjusting agents, preservatives, sweetening agents, crystal growth inhibitors, buffering agents, complexing agents, thickening agents, stabilizers, solubilizers, anti-oxidants, flavoring agents, coloring agents or a combination thereof;
   wherein the pH of the composition is in range of about 3 to about 6.
Embodiment 95. A stable oral liquid pharmaceutical composition comprising:
   (a) about 0.001% to about 0.5% w/w of fingolimod or a pharmaceutically acceptable salt thereof;
   (b) about 10% to 99.99% w/w of a pharmaceutically acceptable vehicle;
   (c) at least one preservative;
   (d) at least one pH adjusting agent; and
   (e) optionally at least one pharmaceutically acceptable excipient selected from sweetening agents, crystal growth inhibitors, buffering agents, complexing agents, thickening agents, stabilizers, solubilizers, anti-oxidants, flavoring agents, coloring agents or a combination thereof;
   wherein the pH of the composition ranges from about 3 to about 6; and
   wherein said composition is stable for at least 6 months when stored at about 25° C. and about 60% relative humidity (RH).
Embodiment 96. A method for treating multiple sclerosis in a patient in need thereof, comprises administering to a patient in need thereof an oral liquid pharmaceutical composition comprising:
   (a) therapeutically effective amount of fingolimod or a pharmaceutically acceptable salt thereof;
   (b) a pharmaceutically acceptable liquid vehicle selected from group consisting of water, ringer's solution, isotonic sodium chloride solution, glycerin, propylene glycol, ethanol, polyethylene glycol or combination thereof;
   (c) optionally one or more excipients selected from group consisting of pH adjusting agents, preservatives, sweetening agents, crystal growth inhibitors, buffering agents, complexing agents, thickening agents, stabilizers, solubilizers, anti-oxidants, flavoring agents, coloring agents or combination thereof;
   wherein the pH of said composition ranges from about 3.0 to about 6.0; and
   wherein said composition is stable for at least 6 months when stored at about 25° C. and about 60% relative humidity (RH).

### EXAMPLES

Abbreviations

| | |
|---|---|
| BQL | below quantifiable limit |
| HDPE | high-density polyethylene |
| HPLC | high-performance liquid chromatography |
| ICH | International Council for Harmonisation |
| ND | not determined |
| NMT | not more than |
| PP | polypropylene |
| q.s. | quantum satis (i.e., quantity sufficient) |
| RH | relative humidity |
| RTU | ready to use |
| USP | United States Pharmacopoeia |

### Analytical Methods

### Stability Study

In the stability studies, a solution was packed in a translucent HDPE bottle with a PP cap and was subjected to the accelerated stability condition (40° C./75% RH or at 60° C. for one month).

### Freeze-Thaw Study

In the freeze-thaw studies, a clear glass vial containing a fingolimod liquid formulation (0.1 mg/mL) was subjected to three to five freeze-thaw cycles, each consisting of 48 hours at −20° C., followed by 48 hours at room temperature.

### Photostability Study

In the freeze-thaw studies, a photostability study was performed by exposing the solution in transparent glass vial to light providing an overall illumination of at least 1.2 million lux hours and an integrated near ultraviolet energy of at least 200-watt hours/square meter.

### Related Substances Assay by HPLC

Related substances were measuring using HPLC as outlined below:

| Instrument | HPLC with PDA/UV detector | | |
|---|---|---|---|
| Column | PRONTOSIL 120-3-C18 ACE-EPS, 150 × 3.0 mm, | | |
| | 3 μm | | |
| Pump mode | Gradient | | |
| Flow rate | 0.8 mL/min | | |
| Detector wavelength | 215 nm | | |
| Column oven | 40° C. | | |
| temperature | | | |
| Sample cooler | 25° C. | | |
| temperature | | | |
| Injection volume | 50 μL | | |
| Run time | 100 min. | | |
| Preparation of | 0.1% Perchloric acid buffer. * | | |
| Mobile phase-A | | | |
| Preparation of | Acetonitrile:Water (80:20 V/V) | | |
| Mobile phase-B | | | |
| *Preparation of | Transfer 1.0 mL of Perchloric acid into 1000 mL of | | |
| buffer | water and mix well. | | |
| Pump mode | Time (min) | % Mobile | % Mobile |
| (Gradient) | | phase-A | phase-B |
| | 0 | 80 | 20 |
| | 80 | 10 | 90 |
| | 90 | 10 | 90 |
| | 91 | 80 | 20 |
| | 100 | 80 | 20 |

### Assay of Fingolimod and Preservative Content by HPLC

Fingolimod and preservative content were measuring used HPLC as outlined below:

| Instrument | HPLC with PDA/UV detector | | | |
|---|---|---|---|---|
| Column | Inertsil ODS 3 V, 150 × 4.6 mm, 3 μm | | | |
| Pump mode | Gradient | | | |
| Flow rate | 0.8 mL/min | | | |
| Detector wavelength | 215 nm | | | |
| Column oven temperature | 40° C. | | | |
| Sample cooler | 25° C. | | | |
| temperature | | | | |
| Injection volume | 10 μL | | | |
| Run time | 40 min. | | | |
| Preparation of | 0.1% Ortho Phosphoric acid buffer*. | | | |
| Mobile phase-A | | | | |
| Preparation of | Acetonitrile:Water (80:20 V/V) | | | |
| Mobile phase-B | | | | |
| Preparation of | 100% Acetonitrile | | | |
| Mobile Phase-C | | | | |
| *Preparation of | Transfer 1.0 mL of Ortho Phosphoric acid | | | |
| buffer | into 1000 mL of water and mix well. | | | |
| Pump mode | Time | % Mobile | % Mobile | % Mobile |
| (Gradient) | (min) | phase-A | phase-B | phase-C |
| | 0 | 70 | 30 | 0 |
| | 25 | 10 | 90 | 0 |
| | 25.1 | 0 | 0 | 100 |
| | 34 | 0 | 0 | 100 |
| | 35 | 70 | 30 | 0 |
| | 40 | 70 | 30 | 0 |

### Example 1. Preparation of Formulation with Methyl Paraben and Propyl Paraben as Preservatives

TABLE 1
Composition of Fingolimod Solution 0.1 mg/mL - Formulation 1
Formulation 1

| Sr. No. | Ingredients | mg/mL | g/1000 mL | % w/w |
|---|---|---|---|---|
| 1 | Fingolimod Hydrochloride | 0.112 | 0.112 | 0.0112 |
| 2 | Sucralose | 0.200 | 0.200 | 0.0200 |
| 3 | Methyl Paraben | 2.000 | 2.000 | 0.2000 |
| 4 | Propyl Paraben | 0.200 | 0.200 | 0.0200 |
| 5 | Purified water | 997.488 | 997.488 | 99.7488 |

| | | | |
|---|---|---|---|
| Total | 1 mL | 1000.00 | 100.00 |

### Manufacturing Procedure

In a clean glass beaker, approximately 900 g of purified water was added, and the solution was heated to 85° C. Sucralose, Methyl Paraben, and Propyl Paraben were added and the mixture was stirred at 85° C. until a clear solution was obtained, followed by cooling to below 30° C. Fingolimod Hydrochloride was then added and stirred to form a clear solution. Volume adjustment was done up to 1000 mL in a volumetric flask. The solution was transferred to a glass beaker and stirred for 10 minutes. The final pH of the clear solution was observed to be 5.53.

The solution was completely frozen at −20° C. and, after keeping at room temperature, a clear solution was observed. There was no precipitation of fingolimod, and the solution was free from any particles. The testing of the solution was performed after three and five freeze-thaw cycles.

The ICH stability, photostability of the fresh sample, and freeze-thaw study data are tabulated in Tables 2a-b, below.

TABLE 2a
ICH Stability, Photostability and Freeze-Thaw Study Data
Formulation 1

| | | 6 Months | 6 Months | 12 Months |
|---|---|---|---|---|
| | | 40° C./ | 25° C./ | 25° C./ |
| Test with Specification | T⁰ | 75% RH | 60% RH | 60% RH |
| Description (A clear, | Complies | Complies | Complies | Complies |
| colorless solution, free | | | | |
| from visible particles) | | | | |
| pH (4.0 to 6.5) | 5.74 | 4.75 | 5.16 | 5.02 |
| Assay of Fingolimod | 99.0 | 101.1 | 100.6 | 100.8 |
| (free base) (90.0 to | | | | |
| 110.0%) | | | | |
| Assay of Methyl | 101.7 | 100.8 | 101.8 | 98.8 |
| paraben (70.0 to | | | | |
| 110.0%) | | | | |
| Assay of Propyl | 101.4 | 95.0 | 99.1 | 96.8 |
| paraben (70.0 to | | | | |
| 110.0%) | | | | |

Related Substances

| | | | | |
|---|---|---|---|---|
| Impurity A (NMT 1.0%) | ND | 0.05 | 0.01 | BQL |
| Mono O-acetyl | ND | ND | ND | ND |
| Fingolimod (NMT 1.0%) | | | | |
| Any individual | 0.024 | ND | ND | 0.01 |
| unspecified impurity | (RRT | | | |
| (NMT 1.0%) | 1.18) | | | |
| Total Impurities (2.0%) | 0.05 | 0.06 | 0.02 | 0.02 |

TABLE 2b
ICH Stability, Photostability and Freeze-Thaw Study Data (continued)
Formulation 1

| | | | Freeze- | Freeze- |
|---|---|---|---|---|
| | | Photo- | Thaw | Thaw |
| | 1 Month | stability | Study | Study |
| Test with Specification | 60° C. | Study | (3 cycles) | (5 cycles) |
| Description (A clear, | Complies | Complies | Complies | Complies |
| colorless solution, | | | | |
| free from visible | | | | |
| particles) | | | | |
| pH (4.0 to 6.5) | 5.89 | 6.07 | 5.88 | 5.50 |
| Assay of Fingolimod | 99.1 | 99.0 | 99.4 | 101.1 |
| (free base) (90.0 to | | | | |
| 110.0%) | | | | |
| Assay of Methyl | 102.0 | 103.3 | 102.5 | 101.1 |
| paraben (70.0 to | | | | |
| 110.0%) | | | | |
| Assay of Propyl | 95.4 | 103.5 | 102.5 | 100.2 |
| paraben (70.0 to | | | | |
| 110.0%) | | | | |

Related Substances

| | | | | |
|---|---|---|---|---|
| Impurity A (NMT | ND | ND | ND | ND |
| 1.0%) | | | | |
| Mono O-acetyl | ND | ND | ND | ND |
| Fingolimod (NMT | | | | |
| 1.0%) | | | | |
| Any individual | 0.057 | 0.036 | 0.02 | 0.33 |
| unspecified impurity | (RRT | (RRT | (RRT | (RRT |
| (NMT 1.0%) | 1.18) | 1.18) | 1.18) | 1.18) |
| Total Impurities | 0.07 | 0.07 | 0.024 | 0.04 |
| (2.0%) | | | | |

As shown above, Formulation 1 is stable at accelerated stability conditions up to 6 months and in long term conditions it is stable up to 12 months. The solution did not get precipitated even after five freeze-thaw cycles. It also shows satisfactory assay and impurity data. The solution is photostable and does not require further protection from the light.

### Example 2. Stability of Solutions at Different pH Ranges

TABLE 3
Composition of Fingolimod Solution
0.1 mg/mL - Formulations 2, 3, and 4
Formulations 2, 3, and 4

| Sr. No. | Ingredients | mg/mL | g/1000 mL | % w/w |
|---|---|---|---|---|
| 1 | Fingolimod | 0.112 | 0.112 | 0.0112 |
| | Hydrochloride | | | |
| 2 | Sucralose | 0.200 | 0.200 | 0.0200 |
| 3 | Methyl Paraben | 2.000 | 2.000 | 0.2000 |
| 4 | Propyl Paraben | 0.200 | 0.200 | 0.0200 |
| 5 | Purified water | 997.488 | 997.488 | 99.7488 |

| | | | |
|---|---|---|---|
| Total | 1 mL | 1000 mL | 100.00 |

### Manufacturing Procedure

In a clean glass beaker, approximately 900 g of purified water was added and heated to 85° C. Sucralose, Methyl Paraben, and Propyl Paraben were added and the mixture was stirred at 85° C. until a clear solution was obtained, followed by cooling to below 30° C. Fingolimod Hydrochloride was added, then the solution was stirred to form a clear solution. Volume adjustment was done up to 1000 mL in a volumetric flask. The solution was transferred to a glass beaker and stirred for 10 minutes.

For Formulation 2, pH was adjusted to 4.5-5.0 using 0.1N HCl solution. A clear solution was observed.

For Formulation 3, pH was adjusted to 3.0-3.5 using 0.1N HCl solution. A clear solution was observed.

For Formulation 4, pH was adjusted to 2.0-2.5 using 0.1N HCl solution. A clear solution was observed.

The ICH stability for Formulations 2, 3, and 4 are tabulated in Tables 4a-c, respectively, below.

TABLE 4a
ICH Stability Study Data for Formulation 2

| | Formulation 2 | | | |
|---|---|---|---|---|
| | | 40° C./ | 25° C./ | |
| Test with | | 75% RH | 60% RH | 1 Month |
| Specification | T⁰ | 6 M | 6 M | 60° C. |
| Description (A clear, | Complies | Complies | Complies | Complies |
| colorless solution, free | | | | |
| from visible particles) | | | | |
| pH (4.0 to 6.5) | 4.86 | 4.06 | 4.78 | 4.64 |
| Assay of Fingolimod | 101.9 | 101.4 | 105.3 | 98.9 |
| (free base) (90.0 to | | | | |
| 110.0%) | | | | |
| Assay of Methyl paraben | 102.4 | 99.2 | 104.2 | 99.6 |
| (70.0 to 110.0%) | | | | |
| Assay of Propyl paraben | 101.9 | 92.9 | 100.7 | 95.4 |
| (70.0 to 110.0%) | | | | |

Related Substances

| | | | | |
|---|---|---|---|---|
| Impurity A (NMT 1.0%) | 0.008 | ND | ND | ND |
| Mono O-acetyl | ND | ND | ND | ND |
| Fingolimod (NMT 1.0%) | | | | |
| Any individual | ND | 0.04 | 0.069 | ND |
| unspecified impurity | | | | |
| (NMT 1.0%) | | | | |
| Total Impurities (2.0%) | 0.009 | 0.041 | 0.08 | 0.018 |

TABLE 4b
ICH Stability Study Data for Formulation 3

| | Formulation 3 | | | |
|---|---|---|---|---|
| | | 40° C./ | 25° C./ | |
| Test with | | 75% RH | 60% RH | 1 Month |
| Specification | T⁰ | 6 M | 6 M | 60° C. |
| Description (A clear, | Complies | Complies | Complies | Complies |
| colorless solution, free | | | | |
| from visible particles) | | | | |
| pH (4.0 to 6.5) | 3.29 | 3.08 | 3.12 | 3.39 |
| Assay of Fingolimod | 101.4 | 101.9 | 102.6 | 101.0 |
| (free base) (90.0 to | | | | |
| 110.0%) | | | | |
| Assay of Methyl paraben | 101.8 | 97.3 | 99.6 | 99.0 |
| (70.0 to 110.0%) | | | | |
| Assay of Propyl paraben | 101.3 | 90.9 | 96.9 | 95.0 |
| (70.0 to 110.0%) | | | | |

Related Substances

| | | | | |
|---|---|---|---|---|
| Impurity A (NMT 1.0%) | ND | ND | ND | ND |
| Mono O-acetyl | ND | ND | ND | ND |
| Fingolimod (NMT 1.0%) | | | | |
| Any individual | ND | 0.03 | 0.002 | ND |
| unspecified impurity | | | | |
| (NMT 1.0%) | | | | |
| Total Impurities (2.0%) | 0.011 | 0.064 | 0.01 | 0.003 |

TABLE 4c
ICH Stability Study Data for Formulation 4

| | Formulation 4 | | |
|---|---|---|---|
| | | 1 Month | |
| Test with | | 40° C./ | 1 Month |
| Specification | T⁰ | 75% RH | 60° C. |
| Description (A clear, colorless | Complies | Complies | Complies |
| solution, free from visible particles) | | | |
| pH (4.0 to 6.5) | 2.05 | 2.12 | 2.09 |
| Assay of Fingolimod (free base) | 101.0 | 101.7 | 101.2 |
| (90.0 to 110.0%) | | | |
| Assay of Methyl paraben (70.0 to | 100.8 | 97.4 | 79.9 |
| 110.0%) | | | |
| Assay of Propyl paraben (70.0 to | 100.6 | 97.4 | 77.0 |
| 110.0%) | | | |

Related Substances

| | | | |
|---|---|---|---|
| Impurity A (NMT 1.0%) | ND | ND | ND |
| Mono O-acetyl Fingolimod | ND | ND | ND |
| (NMT 1.0%) | | | |
| Any individual unspecified impurity | ND | ND | ND |
| (NMT 1.0%) | | | |
| Total Impurities (2.0%) | 0.011 | 0.01 | 0.007 |

### Example 3. Preparation of Formulation with Reduced Levels of Methyl Paraben and Propyl Paraben for Antimicrobial Efficacy Testing

TABLE 5
Composition of Fingolimod Oral Solution 0.1 mg/mL - Formulation 5a
Formulation 5a

| Sr. No. | Ingredients | mg/mL | g/2000 mL | % w/w |
|---|---|---|---|---|
| 1 | Fingolimod Hydrochloride | 0.112 | 0.224 | 0.0112 |
| 2 | Sucralose | 0.200 | 0.400 | 0.0200 |
| 3 | Methyl Paraben | 2.000 | 4.000 | 0.2000 |
| 4 | Propyl Paraben | 0.200 | 0.400 | 0.0200 |
| 5 | 0.1N HCl (for pH | q.s. | q.s. | — |
| | adjustment) | | | |
| 6 | Purified water | 997.488 | 1994.976 | 99.7488 |

| | | | |
|---|---|---|---|
| Total | 1 mL | 2000 mL | 100.00 |

### Manufacturing Procedure

In a clean glass beaker, approximately 1800 g of purified water was added, then heated to 85° C. Sucralose, Methyl Paraben and Propyl Paraben were added and the mixture was stirred at 85° C. until a clear solution was obtained, followed by cooling to below 30° C. After cooling, the pH of the solution was adjusted to 4.5 using a 0.1 N HCl solution. After pH adjustment, Fingolimod Hydrochloride was added, then the solution was stirred until a clear solution was observed. Volume adjustment was done up to 2000 mL in a volumetric flask. The solution was transferred to a glass beaker and stirred for 10 minutes, then filtered through a 5 microns PP (Polypropylene) prefilter. The final solution was observed to be clear at a pH of 4.38.

TABLE 6
Composition of Fingolimod Solution 0.1 mg/mL - Formulation 5b
Formulation 5b

| Sr. No. | Ingredients | mg/mL | g/500 mL | % w/w |
|---|---|---|---|---|
| 1 | Fingolimod Hydrochloride | 0.112 | 0.056 | 0.0112 |
| 2 | Sucralose | 0.200 | 0.100 | 0.0200 |
| 3 | Methyl Paraben | 1.000 | 0.500 | 0.1000 |
| 4 | Propyl Paraben | 0.100 | 0.050 | 0.0100 |
| 5 | 0.1N HCl (for pH adjustment) | q.s. | q.s. | — |
| 6 | Purified water | 998.588 | 499.294 | 99.8588 |

| | | | |
|---|---|---|---|
| Total | 1 mL | 500 mL | 100.00 |

### Manufacturing Procedure

In a clean glass beaker, approximately 450 g of purified water was added, then heated to 85° C. Sucralose, Methyl Paraben, and Propyl Paraben were added and the mixture was stirred at 85° C. until a clear solution was obtained, followed by cooling to below 30° C. Fingolimod Hydrochloride was added, then the solution was stirred until a clear solution was observed. Volume adjustment was done up to 500 mL in a volumetric flask. The solution was transferred to a glass beaker and stirred for 10 minutes. The final pH of the solution was adjusted to 4.5 using a 0.1 N HCl solution, and the final solution was observed to be clear.

### Antimicrobial Efficacy Testing (AET) Data

AET was conducted of Formulations 5a and 5b. The results in Tables 7 and 8, below show the formulations sufficiently prevent the growth of microbial organisms during the testing period.

TABLE 7
Antimicrobial Efficacy Testing (AET) Results for Formulation 5a
Formulation 5a

| | | 14 Days | 28 Days | |
|---|---|---|---|---|
| Organisms | Specifications | Results | Results | Conclusion |
| Burkholderia | At least 1.0 log | 4.27 | No | Conforms the |
| cenocepacia | reduction from | log | increase | specifications |
| | the initial count | reduction | | |
| Burkholderia | at 14 days and no | 4.51 | No | Conforms the |
| cepacia | increase from the | log | increase | specifications |
| | 14 days count at | reduction | | |
| Burkholderia | 28 days | 4.27 | No | Conforms the |
| multivorans | | log | increase | specifications |
| | | reduction | | |
| Escherichia | | >5.79 | No | Conforms the |
| coli | | log | increase | specifications |
| | | reduction | | |
| Pseudomonas | | >5.79 | No | Conforms the |
| aeruginosa | | log | increase | specifications |
| | | reduction | | |
| Staphylococcus | | >5.81 | No | Conforms the |
| aureus | | log | increase | specifications |
| | | reduction | | |
| Candida | No increase from | No | No | Conforms the |
| albicans | the initial | increase | increase | specifications |
| Aspergillus | calculated count | No | No | Conforms the |
| brasiliensis | at 14 days and 28 | increase | increase | specifications |
| | days | | | |

TABLE 8
Antimicrobial Efficacy Testing (AET) Results for Formulation 5a
Formulation 5b

| | | 14 Days | 28 Days | |
|---|---|---|---|---|
| Organisms | Specifications | Results | Results | Conclusion |
| Burkholderia | At least 1.0 log | 4.28 | No | Conforms the |
| cenocepacia | reduction from | log | increase | specifications |
| | the initial count | reduction | | |
| Burkholderia | at 14 days and no | 4.33 | No | Conforms the |
| cepacia | increase from the | log | increase | specifications |
| | 14 days count at | reduction | | |
| Burkholderia | 28 days | 4.36 | No | Conforms the |
| multivorans | | log | increase | specifications |
| | | reduction | | |
| Escherichia | | 4.54 | No | Conforms the |
| coli | | log | increase | specifications |
| | | reduction | | |
| Pseudomonas | | 4.68 | No | Conforms the |
| aeruginosa | | log | increase | specifications |
| | | reduction | | |
| Staphylococcus | | 4.50 | No | Conforms the |
| aureus | | log | increase | specifications |
| | | reduction | | |
| Candida | No increase from | No | No | Conforms the |
| albicans | the initial | increase | increase | specifications |
| Aspergillus | calculated count | No | No | Conforms the |
| brasiliensis | at 14 days and 28 | increase | increase | specifications |
| | days | | | |

### Example 4. Preparation of Formulation with Sodium Propionate as Preservative

TABLE 9
Composition of Fingolimod Solution 0.1 mg/mL-Formulation 6
Formulation 6

| Sr. No. | Ingredients | mg/mL | g/1000 mL | % w/w |
|---|---|---|---|---|
| 1 | Fingolimod Hydrochloride | 0.112 | 0.112 | 0.0112 |
| 2 | Sucralose | 0.200 | 0.200 | 0.0200 |
| 3 | Sodium Propionate | 2.500 | 2.500 | 0.2500 |
| | Anhydrous | | | |
| 4 | 10% HCl | q.s. | q.s. | — |
| | (for pH adjustment) | | | |
| 5 | Purified water | 997.188 | 997.488. | 99.7188 |

| | | | |
|---|---|---|---|
| Total | 1 mL | 1000 mL | 100.00 |

### Manufacturing Procedure

In a clean glass beaker, approximately 900 g of purified water was added. Sucralose and Sodium Propionate were added, and the mixture was stirred until a clear solution was obtained. The pH of the solution was adjusted to 4.0 using 0.1 N HCl solution. Fingolimod Hydrochloride was added, then the solution was stirred until a clear solution was observed. Volume adjustment was done up to 1000 mL in a volumetric flask. The solution was transferred in a glass beaker and stirred for 10 minutes. The final solution was observed to be clear, with the pH of the solution being below 6.5.

### Example 5. Preparation of Formulation with Benzyl Alcohol as Preservative

TABLE 10
Composition of Fingolimod Solution 0.1 mg/mL-Formulation 7
Formulation 7

| Sr. No. | Ingredients | mg/mL | g/1250 mL | % w/w |
|---|---|---|---|---|
| 1 | Fingolimod Hydrochloride | 0.112 | 0.140 | 0.0112 |
| 2 | Sucralose | 0.200 | 0.250 | 0.0200 |
| 3 | Benzyl Alcohol | 5.000 | 6.25 | 0.5000 |
| 4 | 10% HCl | q.s. | q.s. | — |
| | (for pH adjustment) | | | |
| 5 | Purified water | 994.688 | 1243.36 | 99.4688 |

| | | | |
|---|---|---|---|
| Total | 1 mL | 1250 mL | 100.00 |

### Manufacturing Procedure

In a clean glass beaker, approximately 1200 g of purified water was added. Sucralose and Benzyl alcohol were added, and the mixture was stirred until a clear solution was obtained. The pH of the solution was adjusted to 4.0 using 10% HCl solution. Fingolimod Hydrochloride was added, and then the solution was stirred until a clear solution was observed. Volume adjustment was done up to 1250 g in a volumetric flask. The solution was then transferred to a glass beaker and stirred for 10 minutes. The final solution was observed to be clear, with the pH of the solution being below 6.5.

### Example 6. Preparation of Formulation with Sodium Propionate and Benzyl Alcohol as Preservative

TABLE 11
Composition of Fingolimod Solution 0.1 mg/mL-Formulation 8
Formulation 8

| Sr. | | | | |
|---|---|---|---|---|
| No. | Ingredients | mg/mL | g/1000 mL | % w/w |
| 1 | Fingolimod Hydrochloride | 0.112 | 0.112 | 0.0112 |
| 2 | Sucralose | 0.200 | 0.200 | 0.0200 |
| 3 | Sodium Propionate Anhydrous | 2.500 | 2.500 | 0.2500 |
| 4 | Benzyl Alcohol | 15.000 | 15.000 | 1.5000 |
| 5 | 10% HCl (for pH adjustment) | q.s. | q.s. | — |
| 6 | Purified water | 982.188 | 982.188 | 98.2188 |

| | | | |
|---|---|---|---|
| Total | 1 mL | 1000 mL | 100.00 |

### Manufacturing Procedure

In a clean glass beaker, approximately 950 g of purified water was added. Sucralose, Sodium Propionate, and Benzyl alcohol were added, and the mixture was stirred until a clear solution was obtained. The pH of the solution was adjusted to 4.0 using 10% HCl solution. Fingolimod Hydrochloride was added, and the solution was stirred until a clear solution was observed. Volume adjustment was done up to 1000 g in a volumetric flask. The solution was then transferred in a glass beaker and stirred for 10 minutes. The final solution was clear, with the pH of the solution being below 6.5.

### Example 7. Preparation of Formulation with Sodium Benzoate as Preservative

TABLE 12
Composition of Fingolimod Solution-Formulation 9
Formulation 9

| Sr. No. | Ingredients | g/150 mL | % w/v |
|---|---|---|---|
| 1 | Fingolimod Hydrochloride | 0.0224 | 0.0112 |
| 2 | Sucralose | 0.040 | 0.0200 |
| 3 | Grape Flavor | 0.040 | 0.0200 |
| 4 | Sodium Benzoate | 0.400 | 0.2000 |
| 5 | 10% HCl (for pH adjustment) | q.s. | — |
| 6 | Purified water | 150 | 99.7488 |

| | | |
|---|---|---|
| Total | 150 mL | 100 |

### Manufacturing Procedure

In a clean glass beaker, approximately 150 g of purified water was added. Sucralose, Sodium Benzoate, and Grape flavor were added, and the mixture was stirred until a clear solution was obtained. The pH of the solution was adjusted to 4.0 using 10% HCl solution. Fingolimod Hydrochloride was then added, under stirring. With the use of sodium benzoate, precipitation of the mixture was observed at every pH range.

### Example 8. Preparation of Formulation with Meglumine as an Alkalizer for pH Adjustment

TABLE 13
Composition of Fingolimod Oral Solution-Formulation 10
Formulation 10

| Sr. No. | Ingredients | g/150 mL | % w/w |
|---|---|---|---|
| 1 | Fingolimod Hydrochloride | 0.0224 | 0.0112 |
| 2 | Sucralose | 0.0400 | 0.0200 |
| 3 | Methyl Paraben | 0.4000 | 0.2000 |
| 4 | Propyl Paraben | 0.0400 | 0.0200 |
| 5 | Meglumine | 0.500 | 0.2500 |
| 6 | Purified water | 150 | 99.4988 |

| | | |
|---|---|---|
| Total | 150 mL | 100.00 |

### Manufacturing Procedure

In a clean glass beaker, approximately 150 g of purified water was added, then heated to 85° C. Sucralose, Methyl Paraben, and Propyl Paraben were added and the mixture was stirred at 85° C. until a clear solution was obtained, followed by cooling to below 30° C. Meglumine was added, and the mixture was stirred until a clear solution was obtained. pH of the solution was observed 8.5. Fingolimod Hydrochloride was then added, under stirring, and immediate precipitation was observed at a pH above 6.5.

### Example 9. Preparation of Formulation with L-Arginine as an Alkalizer for pH Adjustment

TABLE 14
Composition of Fingolimod Solution-Formulation 11
Formulation 11

| Sr. No. | Ingredients | g/150 mL | % w/w |
|---|---|---|---|
| 1 | Fingolimod Hydrochloride | 0.0224 | 0.0112 |
| 2 | Sucralose | 0.0400 | 0.0200 |
| 3 | Methyl Paraben | 0.4000 | 0.2000 |
| 4 | Propyl Paraben | 0.0400 | 0.0200 |
| 5 | L-Arginine | 0.500 | 0.2500 |
| 6 | Purified water | 150 | 99.4988 |

| | | |
|---|---|---|
| Total | 150 mL | 100 |

### Manufacturing Procedure

In a clean glass beaker, approximately 150 g of purified water was added, then heated to 85° C. Sucralose, Methyl Paraben, and Propyl Paraben were added and the mixture was stirred at 85° C. until a clear solution was obtained, followed by cooling to below 30° C. L-arginine was then added, and the mixture was stirred until a clear solution was obtained. The pH of the solution was observed to be 8.53. Fingolimod Hydrochloride was then added, under stirring, and immediate precipitation was observed at a pH of above 6.5.

### Example 10. Preparation of Formulation with pH Adjustment with 0.1 N HCl and 0.1 N NaOH

TABLE 15
Composition of Fingolimod Solution 0.1 mg/mL-Formulation 12
Formulation 12

| Sr. | | | | |
|---|---|---|---|---|
| No. | Ingredients | mg/mL | g/500 mL | % w/w |
| 1 | Fingolimod Hydrochloride | 0.112 | 0.056 | 0.0112 |
| 2 | Sucralose | 0.200 | 0.1000 | 0.0200 |
| 3 | Methyl Paraben | 2.000 | 1.0000 | 0.2000 |
| 4 | Propyl Paraben | 0.200 | 0.1000 | 0.0200 |
| 5 | 0.1 N HCl (for pH adjustment) | q.s. | q.s. | — |
| 6 | 0.1 N NaOH (for pH adjustment) | q.s. | q.s. | — |
| | Purified water | 997.488 | 498.744 | 99.7488 |

| | | | |
|---|---|---|---|
| Total | 1 mL | 500 mL | 100.00 |

### Manufacturing Procedure

In a clean glass beaker, approximately 450 g of purified water was added, then heated to 85° C. Sucralose, Methyl Paraben, and Propyl Paraben were added and the mixture was stirred at 85° C. until a clear solution was obtained, followed by cooling to below 30° C. The pH of solution was observed to be 5.15. The pH of the solution was then adjusted to 3.5 using a 0.1 N HCl solution and increased to 4.5 using a 0.1 NaOH solution. Fingolimod Hydrochloride was then added, under stirring. Volume adjustment was done up to 500 mL with purified water. A clear solution was observed, with the adjustment of pH using HCl and NaOH, up to a pH of 6.5.

### Example 11. Preparation of Formulation with pH Adjustment Above pH 6.5 with 0.1 N NaOH

TABLE 16
Composition of Fingolimod Solution 0.1 mg/mL-Formulation 13
Formulation 13

| Sr. | | | | |
|---|---|---|---|---|
| No. | Ingredients | mg/mL | g/500 mL | % w/w |
| 1 | Fingolimod Hydrochloride | 0.112 | 0.056 | 0.0112 |
| 2 | Sucralose | 0.200 | 0.1000 | 0.0200 |
| 3 | Methyl Paraben | 2.000 | 1.0000 | 0.2000 |
| 4 | Propyl Paraben | 0.200 | 0.1000 | 0.0200 |
| 5 | 0.1 N NaOH (for pH adjustment) | q.s. | q.s. | — |
| | Purified water | 997.488 | 498.744 | 99.7488 |

| | | | |
|---|---|---|---|
| Total | 1 mL | 500 mL | 100.00 |

### Manufacturing Procedure

In a clean glass beaker, approximately 450 g of purified water was added, then heated to 85° C. Sucralose, Methyl Paraben, and Propyl Paraben were added, and the mixture was stirred at 85° C. until a clear solution was obtained, followed by cooling to below 30° C. Fingolimod Hydrochloride was then added, and the mixture was stirred until a clear solution was obtained. The pH of the solution was then adjusted to 6.0 using a 0.1 NaOH solution, resulting in a clear solution. Volume adjustment was done up to 500 mL with purified water. The pH was then increased by adding a 0.1 NaOH solution. Above a pH 6.5, the mixture formed a precipitate.

### Example 12. Preparation of Formulation with pH Adjustment with 0.1 N HCl and 0.1 N Tri-Sodium Citrate Dihydrate

TABLE 17
Composition of Fingolimod Solution-Formulation 14
Formulation 14

| Sr. No. | Ingredients | g/450 mL | % w/w |
|---|---|---|---|
| 1 | Fingolimod Hydrochloride | 0.056 | 0.0112 |
| 2 | Sucralose | 0.1000 | 0.0200 |
| 3 | Methyl Paraben | 1.0000 | 0.2000 |
| 4 | Propyl Paraben | 0.1000 | 0.0200 |
| 5 | 0.1 N HCl (for pH adjustment) | q.s. | — |
| 6 | 0.1 N Tri-Sodium Citrate Dihydrate | q.s. | — |
| | (for pH adjustment) | | |
| | Purified water | 450 | 99.7488 |

| | | |
|---|---|---|
| Total | 450 mL | 100 |

### Manufacturing Process

In a clean glass beaker, approximately 450 g of purified water was added, then heated to 85° C. Sucralose, Methyl Paraben, and Propyl Paraben were added and the mixture was stirred at 85° C. until a clear solution was obtained, followed by cooling to below 30° C. The pH of the solution was observed to be 5.15. The pH of the solution was then adjusted to 3.5 using a 0.1 N HCl solution, then increased to 4.5 using a 0.1 N Tri-Sodium Citrate Dihydrate solution. Fingolimod Hydrochloride was then added, under stirring, and immediate precipitation was observed at any pH range.

### Example 13. Preparation of Formulation with Potassium Sorbate as Preservative

TABLE 18
Composition of Fingolimod Solution-Formulation 15
Formulation 15

| Sr. No. | Ingredients | g/140 mL | % w/w |
|---|---|---|---|
| 1 | Fingolimod Hydrochloride | 0.0224 | 0.0112 |
| 2 | Xanthan gum | 0.2500 | 0.1250 |
| 3 | Potassium Sorbate | 0.3600 | 0.1800 |
| 4 | Xylitol | 20.0000 | 10.0000 |
| 5 | Sucralose | 0.6000 | 0.3000 |
| 6 | Grape Flavor | 0.4000 | 0.2000 |
| 7 | Purified water | 140 | 89.1838 |

| | | |
|---|---|---|
| Total | 140 mL | 100 |

### Manufacturing Procedure

In a clean glass beaker, approximately 140 g of purified water was added. Sucralose, Xylitol, Xanthan gum, and Potassium sorbate were added, and the mixture was stirred until a clear solution was obtained. The pH of the solution was observed to be 7.35. Fingolimod Hydrochloride was then added, under stirring, and immediate precipitation was observed at pH 7.35.

### Example 14. Preparation of Formulation with Hypromellose

TABLE 19
Composition of Fingolimod Oral Solution 0.1 mg/mL-Formulation 16
Formulation 16

| Sr. | | | | |
|---|---|---|---|---|
| No. | Ingredients | mg/mL | g/1000 mL | % w/w |
| 1 | Fingolimod Hydrochloride | 0.112 | 0.1120 | 0.0112 |
| 2 | HPMC E5 | 10.000 | 10.0000 | 1.0000 |
| 3 | Sucralose | 0.200 | 0.2000 | 0.0200 |
| 4 | Grape Flavor | 0.200 | 0.2000 | 0.0200 |
| 5 | Methyl Paraben | 2.000 | 2.0000 | 0.2000 |
| 6 | Propyl Paraben | 0.200 | 0.2000 | 0.0200 |
| 7 | Purified water | 987.288 | 987.2880 | 98.7288 |

| | | | |
|---|---|---|---|
| Total | 1 mL | 1000 mL | 100.00 |

### Manufacturing Procedure

In a clean glass beaker, approximately 950 g of purified water was added, then heated to 85° C. Methyl Paraben, Propyl Paraben, and Hypromellose were added, and the mixture was stirred at 85° C. until a clear solution was obtained, followed by cooling to below 30° C. Fingolimod Hydrochloride was then added, and the mixture was stirred until a clear solution was observed. Sucralose and Grape flavor were added, then stirred until a clear solution was observed. Volume adjustment was done up to 1000 mL in volumetric flask with purified water. The solution was then stirred for an additional 10 min in a beaker. The solution was observed to be clear at a pH of 6.17.

### Example 15. Preparation of Formulation with Povidone

TABLE 20
Composition of Fingolimod Oral Solution 0.1 mg/mL-Formulation 17
Formulation 17

| Sr. | | | | |
|---|---|---|---|---|
| No. | Ingredients | mg/mL | g/1000 mL | % w/w |
| 1 | Fingolimod Hydrochloride | 0.112 | 0.1120 | 0.0112 |
| 2 | Povidone (PVP K30) | 10.000 | 10.0000 | 1.0000 |
| 3 | Sucralose | 0.200 | 0.2000 | 0.0200 |
| 4 | Grape Flavor | 0.200 | 0.2000 | 0.0200 |
| 5 | Methyl Paraben | 2.000 | 2.0000 | 0.2000 |
| 6 | Propyl Paraben | 0.200 | 0.2000 | 0.0200 |
| 7 | Purified water | 987.288 | 987.2880 | 98.7288 |

| | | | |
|---|---|---|---|
| Total | 1 mL | 1000 mL | 100.00 |

### Manufacturing Procedure

In a clean glass beaker, approximately 950 g of purified water was added, then heated to 85° C. Methyl Paraben, Propyl Paraben, and Povidone were added and the mixture was stirred at 85° C. until a clear solution was obtained, followed by cooling to below 30° C. Fingolimod Hydrochloride was then added, and the mixture was stirred until a clear solution was obtained. Sucralose and Grape flavor were added, and the mixture was again stirred until a clear solution was obtained. Volume adjustment was done up to 1000 mL in volumetric flask with purified water. The solution was stirred for another 10 min in a beaker. The solution was observed to be clear at a pH of 4.09.

### Example 16. Preparation of Formulation with Target pH of 4.5±0.2

TABLE 21
Composition of Fingolimod Oral Solution 0.1 mg/mL-Formulation 18A
Formulation 18A

| Sr. | | | |
|---|---|---|---|
| No. | Ingredients | mg/mL | % w/w |
| 1 | Fingolimod Hydrochloride | 0.112 | 0.0112 |
| 2 | Sucralose | 0.200 | 0.0200 |
| 3 | Methyl Paraben | 2.000 | 0.2004 |
| 4 | Propyl Paraben | 0.200 | 0.0200 |
| 5 | 0.1 N HCl (for pH adjustment) | q.s. | — |
| 6 | 0.1 N NaOH (for pH adjustment) | q.s. | — |
| 7 | Purified water | 995.488 | 99.7483 |

| | | |
|---|---|---|
| Total | 1 mL | 100 mL |

### Manufacturing Procedure

Ensuring availability of hot water in the jacketed mixing tank, the tank was connected to the electric port and the jacket temperature set at 85° C.±5° C. Purified water (97% of the total required quantity) was added to the vessel and heated until the inside temperature reached 85° C. A dispensed quantity of sucralose was added to the vessel with continuous stirring, until a clear solution was observed. The temperature of the solution was maintained at 85° C., and a dispensed quantity of Methyl Paraben and Propyl Paraben were added to the solution, under continuous stirring and mixing, until a clear solution was observed. Stirring was stopped and the temperature of the vessel and the solution were allowed to cool to room temperature by removing the hot water and replacing it with chilled water in the vessel jacket.

The target pH during preparation was 4.5±0.2. If required, the pH of the solution was adjusted using 0.1 N HCl (for lowering the pH) and 0.1 N NaOH (for increasing the pH). After pH adjustment, the solution was kept under continuous stirring, and the dispensed quantity of Fingolimod Hydrochloride was added. Stirring continued until a clear solution was obtained. The solution batch quantity was adjusted using purified water, then the solution was transferred to a holding tank using a cartridge filter.

### Example 17. Preparation of Formulation with Target pH of 4.5±0.2

TABLE 22
Composition of Fingolimod Oral Solution 0.1 mg/mL-Formulation 18B
Formulation 18B

| Sr. | | | | |
|---|---|---|---|---|
| No. | Ingredients | mg/mL | g/5000 mL | % w/w |
| 1 | Fingolimod Hydrochloride | 0.112 | 0.560 | 0.0112 |
| 2 | Sucralose | 0.200 | 1.000 | 0.0200 |
| 3 | Methyl Paraben | 2.000 | 10.000 | 0.2000 |
| 4 | Propyl Paraben | 0.200 | 1.000 | 0.0200 |
| 5 | 0.1 N HCl (for pH adjustment) | q.s to adjust the pH | q.s to adjust the pH | q.s |
| | | to 4.5 | to 4.5 | |
| 6 | 0.1 N NaOH (for pH adjustment) | q.s to adjust | q.s to adjust | q.s |
| | | the pH to 4.5 | the pH to 4.5 | |
| 7 | Purified water | 997.488 | 4987.440 | 99.7488 |

| | | | |
|---|---|---|---|
| Total | 1 mL | 5000 mL | 100.00 |

### Manufacturing Procedure

1. Purified water was taken in a clean vessel and heated to 85° C. temperature.
2. A quantity of sucralose, methyl paraben and propyl paraben were added to above step 1 and stirred at 85° C. temperature until it forms a clear solution followed by cooling below 30° C. temperature.
3. A second quantity of sucralose was added to step 2 and mixed for 5 minutes until a clear solution was formed.
4. A quantity of fingolimod hydrochloride was added to above step 3 and stirred until a clear solution was formed.
5. The pH of the solution was adjusted to 4.5 by using the 0.01% HCl solution and 0.05 N NaOH solution.
6. Volume adjustment was done up to 5000 mL in a volumetric flask. The solution was transferred to a glass beaker and stirred for 10 minutes.
7. The final solution was clear with pH of 4.53.

TABLE 23
Stability Data for Formulation 18B

| | Formulation 18B | | | |
|---|---|---|---|---|
| Test with | | 6 Month | 6 Month | 1 Month |
| Specification | T⁰ | 40° C./75% RH | 25° C./60% RH | 60° C. |
| Description (A clear, colorless | Complies | Complies | Complies | Complies |
| solution, free from visible particles) | | | | |
| pH (3.5 to 6.0) | 4.65 | 4.40 | 4.47 | 4.29 |
| Assay of Fingolimod (free base) | 101.0 | 100.7 | 101.0 | 99.0 |
| Assay of Methyl paraben | 99.2 | 100.0 | 100.9 | 99.2 |
| Assay of Propyl paraben | 99.6 | 97.8 | 100.2 | 96.6 |

Related Substances

| | | | | |
|---|---|---|---|---|
| Impurity A (NMT 1.0%) | ND | 0.02 | ND | ND |
| | | (BLQ) | | |
| Mono O-acetyl Fingolimod | ND | ND | ND | ND |
| (NMT 1.0%) | | | | |
| Any individual unspecified impurity | 0.01 | 0.01 | ND | 0.01 (RRT |
| (NMT 1.0%) | | (BLQ) | | 0.784) |
| Total Impurities | 0.0 | 0.0 | 0.0 | 0.03 |

### EQUIVALENTS

The foregoing written specification is considered to be sufficient to enable one skilled in the art to practice the embodiments. The foregoing description and Examples detail certain embodiments and describe the best mode contemplated by the inventors. It will be appreciated, however, that no matter how detailed the foregoing may appear in text, the embodiment may be practiced in many ways and should be construed in accordance with the appended claims and any equivalents thereof.

1. A stable oral liquid pharmaceutical composition comprising:
(a) a therapeutically effective amount of fingolimod or a pharmaceutically acceptable salt thereof;
(b) a pharmaceutically acceptable liquid vehicle selected from group consisting of water, ringer's solution, isotonic sodium chloride solution, glycerin, propylene glycol, ethanol, and polyethylene glycol or a combination thereof;
(c) optionally one or more excipients selected from group consisting of pH adjusting agents, preservatives, sweetening agents, crystal growth inhibitors, buffering agents, complexing agents, thickening agents, stabilizers, solubilizers, anti-oxidants, flavoring agents, and coloring agents or a combination thereof,
wherein the pH of said composition ranges from about 3.0 to about 6.0; and
wherein said composition is stable for at least 6 months when stored at about 25° C. and about 60% relative humidity (RH).

2. The oral liquid pharmaceutical composition according to claim 1, wherein the composition is a solution, a suspension or an emulsion.

3. The oral liquid pharmaceutical composition according to claim 1, wherein the composition is an aqueous solution.

4. The oral liquid pharmaceutical composition according to claim 1, wherein the pH adjusting agent is selected from HCl and NaOH or a combination thereof.

5. The oral liquid pharmaceutical composition according to claim 1, wherein the preservative is in the range of about 0% to about 3% w/w.

6. The oral liquid pharmaceutical composition according to claim 1, wherein the preservative is selected from methyl paraben, propyl paraben, sodium propionate, benzyl alcohol, potassium sorbate, sodium benzoate, butylated hydroxyanisole, butylated hydroxytoluene, chlorobutanol, and phenyl ethyl alcohol or a combination thereof.

7. The oral liquid pharmaceutical composition according to claim 1, wherein the sweetening agent is in the range of about 0% to about 3% w/w.

8. The oral liquid pharmaceutical composition according to claim 1, wherein the sweetening agent is selected from sucralose, trehalose, xylose, dextrose, tagatose, glycerol, lactitol, saccharine or the corresponding sodium, potassium or calcium salt, cyclamate or the corresponding sodium or calcium salt, aspartame, er and acesulfame or the potassium salt thereof or a combination thereof.

9. The oral liquid pharmaceutical composition according to claim 1, wherein the crystal growth inhibitor is in the range of about 0% to about 5% w/w.

10. The oral liquid pharmaceutical composition according to claim 1, wherein the crystal growth inhibitor is selected from hydroxypropyl methylcellulose, microcrystalline cellulose, hydroxypropyl methylcellulose acetate succinate, and povidone or a combination thereof.

11. The oral liquid pharmaceutical composition according to claim 1, wherein the pH adjusting agent is an alkalizer i-s-selected from meglumine and L-arginine or combination thereof.

12. The oral liquid pharmaceutical composition according to claim 1, wherein the buffering agent is in the range of about 0% to about 5% w/w.

13. The oral liquid pharmaceutical composition according to claim 1, wherein the buffering agent is selected from sodium citrate, sodium acetate, sodium phosphate, potassium phosphate, tris, sodium succinate, histidine, glycine, arginine, sodium malate, sodium tartrate, sodium benzoate, gluconic acid, ascorbic acid, and triethanolamine or combination thereof.

14. The oral liquid pharmaceutical composition according to claim 1, wherein the composition is a ready-to-use composition and does not need further dilution prior to administration.

15. The oral liquid pharmaceutical composition according to claim 1, wherein the composition comprises fingolimod or fingolimod hydrochloride at a concentration in the range of about 0.05 mg/mL to about 10 mg/mL.

16. The oral liquid pharmaceutical composition according to claim 1, wherein the composition comprises fingolimod or fingolimod hydrochloride at a concentration of about 0.1 mg/mL.

17. The oral liquid pharmaceutical composition according to claim 1, wherein the composition has a pH of about 3.0 to about 5.0.

18. The oral liquid pharmaceutical composition according to claim 1, wherein the composition has a pH of about 4.5.

19. The oral liquid composition according to claim 1, wherein the composition is stable after at least two freeze-thaw cycles.

20. The oral liquid pharmaceutical composition according to claim 1, wherein the level of total impurities in the composition is less than about 1% w/w as measured by HPLC, when said solution is stored at about 40° C. and 75% relative humidity (RH) for at least 6 months.

21. A stable oral liquid pharmaceutical composition comprising:
(a) about 0.001% to about 0.5% w/w of fingolimod or a pharmaceutically acceptable salt thereof;
(b) about 10% to 99.99% w/w of a pharmaceutically acceptable vehicle; and
(c) optionally one or more excipients selected from group consisting of pH adjusting agents, preservatives, sweetening agents, crystal growth inhibitors, buffering agents, complexing agents, alkalizers, thickening agents, stabilizers, solubilizers, anti-oxidants, flavoring agents, and coloring agents or a combination thereof;
wherein the pH of the composition is in range of about 3 to about 6.

22. A stable oral liquid pharmaceutical composition comprising:
(a) about 0.001% to about 0.5% w/w of fingolimod or a pharmaceutically acceptable salt thereof;
(b) about 10% to 99.99% w/w of a pharmaceutically acceptable vehicle;
(c) at least one preservative;
(d) at least one pH adjusting agent; and
(e) optionally at least one pharmaceutically acceptable excipient selected from sweetening agents, crystal growth inhibitors, buffering agents, complexing agents, thickening agents, stabilizers, solubilizers, anti-oxidants, flavoring agents, and coloring agents or a combination thereof;
wherein the pH of the composition ranges from about 3 to about 6; and
wherein the composition is stable for at least 6 months when stored at about 25° C. and about 60% relative humidity (RH).

23. A method for treating multiple sclerosis in a patient in need thereof, comprises administering to the patient in need thereof an a stable oral liquid pharmaceutical composition comprising:
(a) a therapeutically effective amount of fingolimod or a pharmaceutically acceptable salt thereof;
(b) a pharmaceutically acceptable liquid vehicle selected from group consisting of water, ringer's solution, isotonic sodium chloride solution, glycerin, propylene glycol, ethanol, and polyethylene glycol or a combination thereof;
(c) optionally one or more excipients selected from group consisting of pH adjusting agents, preservatives, sweetening agents, crystal growth inhibitors, buffering agents, complexing agents, thickening agents, stabilizers, solubilizers, anti-oxidants, flavoring agents, and coloring agents or a combination thereof,
wherein the pH of said composition ranges from about 3.0 to about 6.0; and
wherein said composition is stable for at least 6 months when stored at about 25° C. and about 60% relative humidity (RH).
